(19) United States
(12) Patent Application Publication
(10) Pub. No.: US 2025/0229258 A1
(43) Pub. Date: Jul. 17, 2025

(54) CATALYST PRECURSOR AND METHOD FOR MANUFACTURING SAME, CATALYST MOLDED ARTICLE AND METHOD FOR MANUFACTURING SAME, METHOD FOR MANUFACTURING CATALYST, METHOD FOR MANUFACTURING alpha,beta-UNSATURATED CARBOXYLIC ACID, AND METHOD FOR MANUFACTURING alpha,beta-UNSATURATED CARBOXYLIC ACID ESTER

(71) Applicant: MITSUBISHI CHEMICAL CORPORATION, Tokyo (JP)

(72) Inventors: Kengo OKADA, Tokyo (JP); Yu KURIHARA, Tokyo (JP); Takuro WATANABE, Tokyo (JP); Yuki KATO, Tokyo (JP)

(73) Assignee: MITSUBISHI CHEMICAL CORPORATION, Tokyo (JP)

(21) Appl. No.: 19/064,349

(22) Filed: Feb. 26, 2025

Related U.S. Application Data
(63) Continuation of application No. PCT/JP2023/031706, filed on Aug. 31, 2023.

(30) Foreign Application Priority Data
Aug. 31, 2022 (JP) 2022-137438
Sep. 1, 2022 (JP) 2022-139188

(51) Int. Cl. B01J 27/199; B01J 6/00; B01J 35/61; B01J 35/63; B01J 37/00; B01J 37/04; C07C 67/44

(52) U.S. Cl. CPC B01J 27/199; B01J 6/001; B01J 35/612; B01J 35/633; B01J 37/0045; B01J 37/04; C07C 67/44

(57) ABSTRACT

Provided is a catalyst precursor containing a Keggin-type heteropolyacid used for the production of an α,β-unsaturated carboxylic acid by oxidation of an α,β-unsaturated aldehyde.

### CROSS REFERENCE TO RELATED APPLICATIONS

This application is a Continuation of PCT International Application No. PCT/JP2023/031706, filed on Aug. 31, 2023, which is claiming priority of Japanese Patent Application No. 2022-137438, filed on Aug. 31, 2022, and Japanese Patent Application No. 2022-139188, filed on Sep. 1, 2022, the entire contents of which are incorporated herein by reference.

### TECHNICAL FIELD

The present invention relates to: a catalyst precursor and a method of producing the same; a molded catalyst product and a method of producing the same; a method of producing a catalyst; a method of producing an α,β-unsaturated carboxylic acid; and a method of producing an α,β-unsaturated carboxylic acid ester.

### BACKGROUND ART

Heteropolyacid-based catalysts such as phosphomolybdic acid are known as catalysts for α,β-unsaturated carboxylic acid production (hereinafter, also simply referred to as “catalysts”) that are used for the production of an α,β-unsaturated carboxylic acid by oxidation of an α,β-unsaturated aldehyde. Numerous studies have been conducted on the method of producing a heteropolyacid-based catalyst and, in many of these studies, a catalyst is produced by first preparing an aqueous solution or slurry that contains the elements constituting the catalyst, subsequently drying the aqueous solution or slurry to produce a catalyst precursor, and then molding and/or calcinating the catalyst precursor. In this process, it is known that the physical properties such as the particle size of the resulting catalyst precursor and catalyst and the specific surface area of the resulting catalyst, are modified and the catalyst performance is affected by the slurry preparation conditions and drying conditions.

Patent Document 1 discloses a method of producing a catalyst for the production of methacrylic acid, which catalyst is used for producing methacrylic acid by gas-phase catalytic oxidation of methacrolein with molecular oxygen and contains at least molybdenum and phosphorus as catalyst components.

Further, in the production of an α,β-unsaturated carboxylic acid, a catalyst is generally molded into a molded article having a spherical shape of about 2 to 20 mm in diameter or a columnar or cylindrical shape of about 2 to 10 mm in diameter and 2 to 20 mm in length, and packed into a reactor as a molded catalyst product. Such a molded catalyst product is known to exhibit varying performance depending on its pore structure, and numerous studies have been conducted on the control of the pore structure of a molded catalyst product.

Patent Document 2 discloses a catalyst used for the production of methacrylic acid, which catalyst has a pore volume of 0.10 to 1.0 cc/g and a pore distribution in which the distribution of pore diameter is concentrated in each range of 1 to 10 μm and 0.1 to 1 μm.

Patent Document 3 discloses a catalyst for the production of methacrylic acid, which catalyst is characterized by having at least two peaks in a pore radius range of 0.5 to 10 μm in a pore size distribution chart.

Patent Document 4 discloses a method of producing a catalyst for the production of methacrylic acid, which catalyst is used for producing methacrylic acid by gas-phase catalytic oxidation of methacrolein with molecular oxygen and contains at least molybdenum and phosphorus as catalyst components, and it is described that, when a ratio between the apparent density of a dried catalyst and the density of a molded catalyst is in a specific range, the yield of methacrylic acid is improved since a pore amount effective for the selective oxidation of methacrolein and a catalyst packing amount effective for the oxidation of methacrolein can both be ensured.

### RELATED ART DOCUMENTS

### Patent Documents

[Patent Document 1] Japanese Unexamined Patent Application Publication No. 2011-224482
[Patent Document 2] Japanese Unexamined Patent Application Publication No. S63-315148
[Patent Document 3] Japanese Unexamined Patent Application Publication No. 2000-84412
[Patent Document 4] WO 2012/141076

### SUMMARY OF THE INVENTION

### Problems to be Solved by the Invention

However, the catalysts disclosed in Patent Documents 1 to 4 are not necessarily sufficient in terms of the α,β-unsaturated carboxylic acid yield. Therefore, from the standpoint of further improving the catalyst performance, it is demanded to develop a catalyst that has physical properties more suitable for the production of an α,β-unsaturated carboxylic acid.

### Means for Solving the Problems

The present inventors intensively studied in view of the above-described problems and consequently discovered that the problems can be solved by using a catalyst precursor having a specific pore volume, or a molded catalyst product having a specific pore volume and a specific pore distribution, thereby completing the present invention.

That is, the present invention encompasses the following.

[1]: A catalyst precursor, containing a Keggin-type heteropolyacid and being used for the production of an α,β-unsaturated carboxylic acid by oxidation of an α,β-unsaturated aldehyde, wherein the catalyst precursor has a pore volume of 0.005 to 0.15 mL/g.

[2]: The catalyst precursor according to [1], having a median diameter of 1 to 50 μm.

[3]: The catalyst precursor according to [2], wherein the median diameter is 5 to 40 μm.

[4]: The catalyst precursor according to any one of [1] to [3], wherein the pore volume is 0.01 to 0.10 mL/g.

[5]: The catalyst precursor according to any one of [1] to [4], having a bulk density of 1.15 to 1.6 kg/L.

[6]: The catalyst precursor according to any one of [1] to [5], having a composition represented by the following Formula (I):

PₐMO_{b}V_{c}Cu_{d}AₑE_{f}G_{g}(NH₄)ₕOᵢ (I)
(wherein, P, Mo, V, Cu, NH₄, and O represent phosphorus, molybdenum, vanadium, copper, ammonium radical, and oxygen, respectively; A represents at least one element selected from the group consisting of antimony, bismuth, arsenic, germanium, tellurium, selenium, silicon, and tungsten; E represents at least one element selected from the group consisting of iron, zinc, chromium, tantalum, cobalt, nickel, manganese, titanium, niobium, and cerium; G represents at least one element selected from the group consisting of lithium, sodium, potassium, rubidium, and cesium; and a to i each represent a molar ratio of the respective component, satisfying b=12, a=0.5 to 3, c=0.01 to 3, d=0.01 to 2, e=0 to 3, f=0 to 3, g=0.01 to 3, and h=1 to 30, with i being a molar ratio of oxygen that is required for satisfying the valences of the respective elements).

[7]: A molded catalyst product, containing a catalyst component that contains phosphorus, molybdenum, and vanadium, and being used for the production of an α,β-unsaturated carboxylic acid by oxidation of an α,β-unsaturated aldehyde,
wherein
the molded catalyst product has a pore volume of 0.01 to 0.40 mL/g, and
in a pore distribution curve of the molded catalyst product, when the height of a highest peak (peak A) and that of a second highest peak (peak B), apexes of which exist in a pore diameter range of 0.05 to 10 μm, are defined as IA and IB, respectively, a ratio IB/IA is 0.160 to 0.420.

[8]: The molded catalyst product according to [7], wherein the ratio IB/IA is 0.200 to 0.400.

[9]: The molded catalyst product according to [7] or [8], having a specific surface area of 1 to 10 m²/g.

[10]: The molded catalyst product according to [9], wherein the specific surface area is 1.5 to 8 m²/g.

[11]: The molded catalyst product according to any one of [7] to [10], wherein the pore volume is 0.10 to 0.35 mL/g.

[12]: The molded catalyst product according to any one of [7] to [11], wherein the apexes of the peak A and the peak B exist in a pore diameter range of 0.08 to 8 μm.

[13]: The molded catalyst product according to any one of [7] to [12], which is an extrusion-molded article.

[14]: A method of producing a catalyst precursor according to any one of [1] to [6], including:
(i) a step of obtaining a solution or slurry (liquid A1) that contains phosphorus, molybdenum, and vanadium;
(ii) a step of adding an ammonium radical-containing raw material compound (compound B) to the liquid A1 to obtain a slurry having a pH of 3 or lower (liquid A2); and
(iii) a step of drying the liquid A2 to obtain dry particles, wherein, in the step (ii), the compound B is added such that the following Formula (II) is satisfied:

$$v / M = 0.1 to 1.8 \qquad ( II )$$

(wherein, M represents the number of moles (mol) of molybdenum contained in the liquid A1, and v represents an ammonium radical addition rate (mol/h)).

[15]: A method of producing a molded catalyst product from a catalyst precursor produced by the method according to [14], including (iv) a step of extrusion-molding the dry particles to obtain a molded catalyst product.

[16]: A method of producing a catalyst, the method including calcinating the catalyst precursor according to any one of [1] to [6], or a catalyst precursor produced by the method according to [14].

[17]: A method of producing a molded catalyst product according to any one of [7] to [13], including:
(i) a step of obtaining a solution or slurry (liquid A1) that contains phosphorus, molybdenum, and vanadium;
(ii) a step of adding an ammonium radical-containing raw material compound (compound B) to the liquid A1 to obtain a slurry having a pH of 3 or lower (liquid A2);
(iii) a step of drying the liquid A2 to obtain dry particles; and
(iv) a step of extrusion-molding the dry particles to obtain a molded catalyst product, wherein, in the step (ii), the compound B is added such that the following Formula (II) is satisfied:

$$v / M = 0.1 to 1.8 \qquad ( II )$$

(wherein, M represents the number of moles (mol) of molybdenum contained in the liquid A1, and v represents an ammonium radical addition rate (mol/h)).

[18]: The method of producing a molded catalyst product according to [15] or [17], wherein the liquid A2 has a solid concentration of 30% by mass or lower.

[19]: The method of producing a molded catalyst product according to any one of [15], [17], and [18], wherein, in the liquid A2, when a ratio of a total mass of dissolved molybdenum element, phosphorus element, and vanadium element with respect to a total mass of molybdenum element, phosphorus element, and vanadium element is defined as R, R is 5 to 25% by mass.

[20]: The method of producing a molded catalyst product according to any one of [15] and [17] to [19], wherein, in the step (ii), the compound B is added while stirring the liquid A1 having a temperature of 90 to 99° C. at a rotation speed of 70 to 140 rpm.

[21]: The method of producing a molded catalyst product according to any one of [15] and [17] to [20], wherein, in the step (iii), the liquid A2 is spray-dried.

[22]: The method of producing a molded catalyst product according to any one of [15] and [17] to [21], wherein the step (iv) includes the following steps (iv-1) and (iv-2):
(iv-1) a step of mixing the dry particles with a liquid and a binder to obtain a kneaded product; and
(iv-2) a step of extrusion-molding the kneaded product using an extruder to obtain a molded catalyst product.

[23]: The method of producing a molded catalyst product according to [22], wherein, in the step (iv-1), 15 to 60 parts by mass of the liquid and 0.05 to 15 parts by mass of the binder are mixed with respect to 100 parts by mass of the dry particles.

[24]: The method of producing a molded catalyst product according to [22] or [23], wherein, in the step (iv-2), the extrusion molding is performed at an extrusion pressure of 0.1 to 30 MPa.

[25]: A method of producing an α,β-unsaturated carboxylic acid, the method including oxidizing an α,β-unsaturated aldehyde using a catalyst obtained by molding and/or calcinating the catalyst precursor according to any one of [1] to [6], or the molded catalyst product according to any one of [7] to [13].

[26]: A method of producing an α,β-unsaturated carboxylic acid ester, the method including esterifying an α,β-unsaturated carboxylic acid produced by the method according to [25].

### Effects of the Invention

According to the present invention, a catalyst precursor from which a catalyst having a high α,β-unsaturated carboxylic acid yield can be produced, or a molded catalyst product can be provided.

### BRIEF DESCRIPTION OF THE DRAWINGS

FIG. 1 shows pore distribution curves of the catalyst precursors obtained in Example 1-1, Example 1-4, and Comparative Example 1-1.

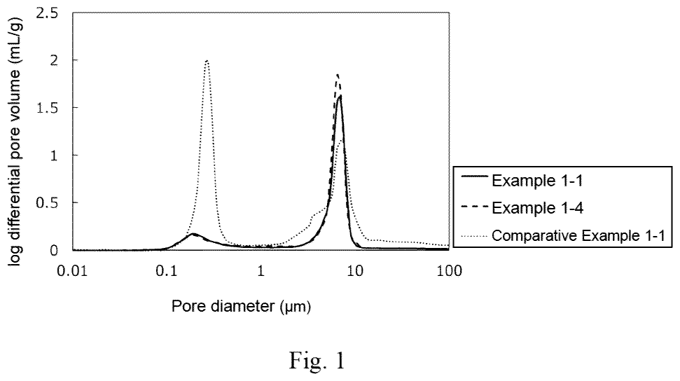

FIG. 2 shows cumulative pore volume curves of the catalyst precursors obtained in Example 1-1, Example 1-4, and Comparative Example 1-1.

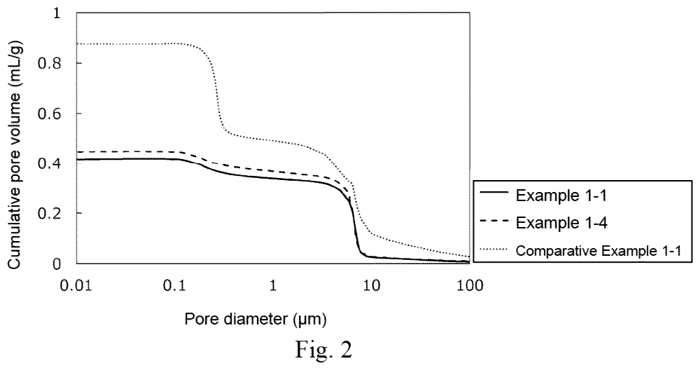

### MODE FOR CARRYING OUT THE INVENTION

Embodiments of the present invention will now be described; however, the present invention is not limited to the below-described embodiments.

In the present specification, those numerical ranges that are expressed with “to” each denote a range that includes the numerical values stated before and after “to” as the lower limit value and the upper limit value, respectively, and an expression “A to B” means a value that is A or more but B or less.

### [Catalyst Precursor]

The catalyst precursor according to the present embodiment is a catalyst precursor used for the production of an α,β-unsaturated carboxylic acid by oxidation of an α,β-unsaturated aldehyde, and the catalyst precursor contains a Keggin-type heteropolyacid and has a pore volume of 0.005 to 0.15 mL/g. By using a catalyst produced from this catalyst precursor, an α,β-unsaturated carboxylic acid can be produced with a high yield.

### <Composition of Catalyst Precursor>

The catalyst precursor according to the present embodiment contains a Keggin-type heteropolyacid. One example of a method of obtaining a catalyst precursor containing a Keggin-type heteropolyacid is a method of producing a catalyst precursor by the below-described production method in which the pH of the liquid A2 is adjusted to be 3 or lower in the step (ii).

Whether or not the resulting catalyst precursor contains a Keggin-type heteropolyacid can be verified by an infrared absorption analysis using NICOLET 6700FT-IR (product name, manufactured by Thermo Electron Corporation) or the like, or an X-ray diffraction analysis using an X-ray diffractometer X'Pert PRO MPD (product name, manufactured by PANalytical Ltd.) or the like.

The catalyst precursor according to the present embodiment preferably contains phosphorus, molybdenum, and vanadium. From the standpoint of improving the α,β-unsaturated carboxylic acid yield, when the number of molybdenum atoms is 12, a ratio of the number of phosphorus atoms is preferably 0.5 to 3, and the lower limit thereof is more preferably 0.6 or higher, still more preferably 0.7 or higher, particularly preferably 0.8 or higher. The upper limit of this ratio is more preferably 2.5 or lower, still more preferably 2 or lower. Further, when the number of molybdenum atoms is 12, a ratio of the number of vanadium atoms is preferably 0.01 to 3, and the lower limit thereof is more preferably 0.05 or higher, still more preferably 0.1 or higher, particularly preferably 0.2 or higher. The upper limit of this ratio is more preferably 2.5 or lower, still more preferably 2 or lower, particularly preferably 1.5 or lower.

From the standpoint of improving the α,β-unsaturated carboxylic acid yield, the catalyst precursor according to the present embodiment particularly preferably has a composition represented by the following Formula (I). The catalyst precursor may also contain a small amount of an element that is not included in the following Formula (I).

PₐMO_{b}V_{c}Cu_{d}AₑE_{f}G_{g}(NH₄)ₕOᵢ (I)

In Formula (I), P, Mo, V, Cu, NH₄, and O represent phosphorus, molybdenum, vanadium, copper, ammonium radical, and oxygen, respectively; A represents at least one element selected from the group consisting of antimony, bismuth, arsenic, germanium, tellurium, selenium, silicon, and tungsten; E represents at least one element selected from the group consisting of iron, zinc, chromium, tantalum, cobalt, nickel, manganese, titanium, niobium, and cerium; G represents at least one element selected from the group consisting of lithium, sodium, potassium, rubidium, and cesium; and a to i each represent a molar ratio of the respective component, satisfying b=12, a=0.5 to 3, c=0.01 to 3, d=0.01 to 2, e=0 to 3, f=0 to 3, g=0.01 to 3, and h=1 to 30, with i being a molar ratio of oxygen that is required for satisfying the valences of the respective elements.

In Formula (I), from the standpoint of improving the activity and the life of a catalyst, G is preferably at least one element selected from the group consisting of potassium and cesium.

In Formula (I), from the standpoint of improving the α,β-unsaturated carboxylic acid yield, the lower limit of a is preferably 0.6 or more, more preferably 0.7 or more, still more preferably 0.8 or more, while the upper limit of a is preferably 2.5 or less, more preferably 2 or less. The lower limit of c is preferably 0.05 or more, more preferably 0.1 or more, still more preferably 0.2 or more, while the upper limit of c is preferably 2.5 or less, more preferably 2 or less, still more preferably 1.5 or less. The lower limit of d is preferably 0.03 or more, more preferably 0.05 or more, still more preferably 0.1 or more, while the upper limit of d is preferably 1.5 or less, more preferably 1 or less. The lower limit of e is preferably 0.01 or more, more preferably 0.1 or more, while the upper limit of e is preferably 2.5 or less, more preferably 2 or less. The lower limit of f is preferably 0.01 or more, more preferably 0.03 or more, while the upper limit of f is preferably 2 or less, more preferably 1.5 or less, still more preferably 1 or less. The lower limit of g is preferably 0.1 or more, more preferably 0.3 or more, still more preferably 0.5 or more, while the upper limit of g is preferably 2.8 or less, more preferably 2.5 or less, still more preferably 2 or less. The lower limit of h is preferably 2 or more, more preferably 3 or more, while the upper limit of h is preferably 20 or less, more preferably 10 or less.

The molar ratio of each element is defined as a value determined by analyzing the components of the catalyst precursor dissolved in aqueous ammonia by ICP emission spectrometry. Further, the molar ratio of ammonium radical is defined as a value determined by analyzing the catalyst precursor by the Kjeldahl method. The term “ammonium radical” used herein is a general term for ammonia (NH₃) and ammonium ion (NH₄⁺)

### <Pore Volume of Catalyst and Catalyst Precursor>

An increase in the pore volume of a catalyst leads to an increase in the specific surface area of the catalyst. As a result, the activity of the catalyst is improved, so that the α,β-unsaturated carboxylic acid yield is improved. On the other hand, an excessively large pore volume of the catalyst leads to a reduction in the packing density of the catalyst, resulting in a reduction in the weight of the catalyst that can be packed into a reactor of a certain volume. Therefore, a sufficient catalytic activity cannot be obtained, and the α,β-unsaturated carboxylic acid yield is reduced. In other words, since both an effect of improving the α,β-unsaturated carboxylic acid yield and an effect of reducing the α,β-unsaturated carboxylic acid yield are obtained by increasing the pore volume, it has been difficult to sufficiently improve the α,β-unsaturated carboxylic acid yield by controlling the pore volume of a catalyst. In view of this, in the present invention, the pore volume of a catalyst that is more suitable for the production of an α,β-unsaturated carboxylic acid was studied.

The catalyst precursor according to the present embodiment has a pore volume of 0.005 to 0.15 mL/g. When the pore volume is 0.005 mL/g or more, a catalyst produced from the catalyst precursor has a sufficiently large specific surface area, so that the α,β-unsaturated carboxylic acid yield is improved. Meanwhile, when the pore volume is 0.15 mL/g or less, a catalyst produced from the catalyst precursor has a sufficiently high packing density, and this leads to an increase in the weight of the catalyst that can be packed into a reactor of a certain volume; therefore, the α,β-unsaturated carboxylic acid yield is improved. The lower limit of the pore volume is preferably 0.01 mL/g or more, more preferably 0.05 mL/g or more, while the upper limit of the pore volume is preferably 0.10 mL/g or less, more preferably 0.09 mL/g or less.

In the present embodiment, the pore volume of the catalyst precursor means the volume of pores inside the particles of the catalyst precursor. The pore volume of the catalyst precursor can be measured using a mercury-intrusion pore distribution analyzer. Examples of the mercury-intrusion pore distribution analyzer include AutoPore IV-9500 (product name, manufactured by Micromeritics Instrument Corporation). It is noted here that, in the pore distribution measurement by a mercury intrusion method, not only the volume of pores inside the particles of the catalyst precursor, which is the pore volume of the catalyst precursor according to the present embodiment, but also the volume of pores derived from physical voids between the particles of the catalyst precursor are measured. The pore volume of the catalyst precursor according to the present embodiment is defined as a value calculated from the volume of pores having a diameter of 0.5 μm or less.

Examples of a method of obtaining a catalyst precursor having a pore volume in the above-described range include a method of producing a catalyst precursor by the below-described production method in which the addition rate v (mol/h) of the compound B, the temperature of the liquid A1, and the stirring rotation speed of the liquid A1 are adjusted in the step (ii), the physical properties of the liquid A2 are adjusted, or the drying conditions are adjusted in the step (iii).

### <Median Diameter of Catalyst Precursor>

The catalyst precursor according to the present embodiment preferably has a median diameter of 1 to 50 μm. When the median diameter of the catalyst precursor is 1 μm or more, an α,β-unsaturated aldehyde used as a raw material and the resulting α,β-unsaturated carboxylic acid can sufficiently diffuse into pores derived from voids between particles of a catalyst produced from the catalyst precursor; therefore, the α,β-unsaturated carboxylic acid yield is improved. Meanwhile, when the median diameter of the catalyst precursor is 50 μm or less, the reaction substrate can easily diffuse into the particles of the catalyst produced from the catalyst precursor; therefore, a high α,β-unsaturated carboxylic acid yield can be obtained even if the pore volume is small. The lower limit of the median diameter is more preferably 5 μm or more, still more preferably 10 μm or more, while the upper limit of the median diameter is more preferably 40 μm or less, still more preferably 30 μm or less.

The term “median diameter” used herein refers to a particle size that corresponds to a cumulative volume of 50% in a volume-based particle size distribution measured by a laser diffraction-type particle size distribution measurement method, and the measurement can be performed using, for example, a laser diffraction-type wet particle size distribution analyzer or a laser diffraction-type dry particle size distribution analyzer. Examples of the laser diffraction-type wet particle size distribution analyzer include LA-700 (product name, manufactured by Horiba, Ltd.), LA-900 (product name, manufactured by Horiba, Ltd.), LA-960V2 (product name, manufactured by Horiba, Ltd.), and SALD-7000 (product name, manufactured by Shimadzu Corporation). Examples of the laser diffraction-type dry particle size distribution analyzer include SALD-2300 (product name, manufactured by Shimadzu Corporation).

Examples of a method of obtaining a catalyst precursor having a median diameter in the above-described range include a method of producing a catalyst precursor by the below-described production method in which the addition rate v (mol/h) of the compound B, the temperature of the liquid A1, and the stirring rotation speed of the liquid A1 are adjusted in the step (ii), the physical properties of the liquid A2 are adjusted, or the drying conditions are adjusted in the step (iii).

### <Bulk Density of Catalyst Precursor>

The catalyst precursor according to the present embodiment preferably has a bulk density of 1.15 to 1.6 kg/L. When the bulk density of the catalyst precursor is 1.15 kg/L or higher, at the time of packing a catalyst produced from the catalyst precursor into a reactor of a certain volume, the weight of the catalyst that can be packed is increased; therefore, a sufficient catalytic activity is obtained in the production of an α,β-unsaturated carboxylic acid. Meanwhile, when the bulk density of the catalyst precursor is 1.6 kg/L or lower, the catalyst produced from the catalyst precursor has a sufficiently large specific surface area, so that the α,β-unsaturated carboxylic acid yield is improved. The lower limit of the bulk density of the catalyst precursor is more preferably 1.2 kg/L or higher, still more preferably 1.25 kg/L or higher. The upper limit of the bulk density of the catalyst precursor is more preferably 1.55 kg/L or lower, still more preferably 1.5 kg/L or lower.

In the present invention, the bulk density of the catalyst precursor is calculated by the following equation from the mass of the catalyst precursor weighed in a graduated cylinder having a volume of 50 mL.

Bulk density (kg/L) of catalyst precursor=Weight (g) of catalyst precursor packed in 50-mL graduated cylinder/50 (mL)

### [Method of Producing Catalyst Precursor]

The catalyst precursor according to the present embodiment, as long as it contains a Keggin-type heteropolyacid and has a pore volume of 0.005 to 0.15 mL/g, can be produced in accordance with any known catalyst precursor production method; however, the catalyst precursor according to the present embodiment is preferably produced by the method of producing a catalyst precursor according to the present embodiment, which method includes the following steps (i) to (iii):
(i) a step of obtaining a solution or slurry (liquid A1) that contains phosphorus, molybdenum, and vanadium;
(ii) a step of adding an ammonium radical-containing raw material compound (compound B) to the liquid A1 to obtain a slurry having a pH of 3 or lower (liquid A2); and
(iii) a step of drying the liquid A2 to obtain dry particles.

These steps will now be described in detail.

<Step (i)>

In the step (i), a solution or slurry (liquid A1) that contains phosphorus, molybdenum, and vanadium is obtained. The liquid A1 may also contain other elements, such as Cu (copper), an A element, an E element, and a G element in the above-described Formula (I). These elements other than phosphorus, molybdenum, and vanadium in Formula (I) may be added in after the step (i).

The liquid A1 can be prepared by dissolving or suspending raw material compounds of catalyst precursor components that contain phosphorus, molybdenum, and vanadium in a solvent.

### (Raw Material Compounds of Catalyst Precursor Components)

The raw material compounds of the catalyst precursor components are not particularly limited, and nitrates, carbonates, bicarbonates, acetates, ammonium salts, oxides, halides, oxoacids, oxoacid salts, and the like of the respective constituent elements of the catalyst precursor may be used singly, or in combination of two or more kinds thereof.

Examples of a raw material compound of phosphorus include phosphoric acid, phosphorus pentoxide, and ammonium phosphate. Examples of a raw material compound of molybdenum include: molybdenum oxides, such as molybdenum trioxide; ammonium molybdates, such as ammonium paramolybdate and ammonium dimolybdate; and molybdenum chloride. Examples of a raw material compound of vanadium include ammonium metavanadate, vanadium pentoxide, vanadium chloride, and vanadyl oxalate.

In the case of producing a catalyst precursor that contains copper in addition to phosphorus, molybdenum, and vanadium, examples of a raw material compound of copper include copper sulfate, copper nitrate, copper oxide, copper carbonate, copper acetate, and copper chloride. In the case of producing a catalyst precursor that contains a G element in addition to phosphorus, molybdenum, and vanadium, examples of a raw material compound of the G element include nitrates, carbonates, bicarbonates, hydroxides, sulfates, acetates, and chlorides, and it is preferred to use a carbonate or a bicarbonate.

The raw material compounds of the catalyst precursor components may be used singly, or in combination of two or more kinds thereof, for each element constituting the catalyst precursor components.

A total concentration of the raw material compounds of the catalyst precursor components in the liquid A1 is not particularly limited; however, it is preferably in a range of 5 to 90% by mass.

### (Solvent)

Examples of the above-described solvent include water, ethyl alcohol, and acetone. These solvents may be used singly, or in combination of two or more kinds thereof. Thereamong, from the industrial standpoint, it is preferred to use water.

### (Preparation of Liquid A1)

The liquid A1 is preferably prepared by adding the raw material compounds of the catalyst precursor components to the solvent in a preparation vessel, and stirring these materials with heating. By this, a heteropolyacid suitable for the production of an α,β-unsaturated carboxylic acid is sufficiently generated.

The heating can be usually performed in a range of 30 to 150° C., and it is preferably performed in a range of 60 to 150° C. The heteropolyacid generation rate can be sufficiently increased by setting the heating temperature at 60° C. or higher, and the evaporation of the solvent can be inhibited by setting the heating temperature at 150° C. or lower. The lower limit of the heating temperature is more preferably 80° C. or higher, still more preferably 90° C. or higher, while the upper limit of the heating temperature is more preferably 130° C. or lower, still more preferably 110° C. or lower. Further, depending on the vapor pressure of the solvent to be used, the materials may be concentrated or refluxed during the heating, or may be heat-treated under a pressurized condition by an operation in a sealed vessel.

The heating rate is not particularly limited; however, it is preferably 0.8 to 15° C./min. When the heating rate is 0.8° C./min or higher, the time required for the step (i) can be shortened. Meanwhile, when the heating rate is 15° C./min or lower, the heating can be performed using an ordinary heating equipment.

The stirring is performed with a stirring power of preferably 0.01 kW/m³ or more, more preferably 0.05 kW/m³ or more. By setting the stirring power at 0.01 kW/m³ or more, local variations in the components and the temperature of the liquid A1 are reduced, so that a structure suitable for the production of an α,β-unsaturated carboxylic acid is stably formed. Further, from the standpoint of the production cost of the catalyst precursor, usually, the stirring is preferably performed with a stirring power of 3.5 kW/m³ or less.

### (Physical Properties of Liquid A1)

The pH of the liquid A1 is not particularly limited; however, it is preferably 0.1 to 4, and the lower limit thereof is more preferably 0.5 or higher, while the upper limit thereof is more preferably 3 or lower. When the pH of the liquid A1 is 0.1 or higher, the below-described step (ii) can be stably performed. Further, when the pH of the liquid A1 is 4 or lower, a Keggin-type heteropolyacid suitable for the production of an α,β-unsaturated carboxylic acid is stably generated. As a method of controlling the pH of the liquid A1 to be 0.1 to 4, for example, molybdenum trioxide may be used as a molybdenum raw material, or the content of nitrate ions or oxalate ions may be adjusted by appropriately selecting a raw material compound. The pH can be measured using a pH meter, such as D-21 (product name, manufactured by Horiba, Ltd.). Further, whether or not a heteropolyacid salt having a Keggin structure has been formed can be verified by an infrared absorption analysis using NICOLET 6700FT-IR (product name, manufactured by Thermo Electron Corporation) or the like, or an X-ray diffraction analysis using an X-ray diffractometer X'Pert PRO MPD (product name, manufactured by PANalytical Ltd.) or the like.

<Step (ii)>

In the step (ii), an ammonium radical-containing raw material compound (compound B) is added to the liquid A1 obtained in the above-described step (i) to obtain a slurry having a pH of 3 or lower (liquid A2).

### (Compound B)

Examples of the compound B include ammonium bicarbonate, ammonium carbonate, ammonium nitrate, aqueous ammonia, ammonium phosphate, and ammonium metavanadate. These compounds may be used singly, or in combination of two or more kinds thereof.

The compound B may be dissolved or suspended in a solvent. Examples of the solvent include water, ethyl alcohol, and acetone. These solvents may be used singly, or in combination of two or more kinds thereof. Thereamong, from the industrial standpoint, it is preferred to use water.

### (Addition of Compound B)

The compound B is preferably added such that the following Formula (II) is satisfied:

$$v / M = 0.1 to 1.8 \qquad ( II )$$

In Formula (II), M represents the number of moles (mol) of molybdenum contained in the liquid A1, and v represents an ammonium radical addition rate (mol/h)).

Satisfaction of Formula (II) means that the ammonium radical addition rate is sufficiently low. By this, the degree of supersaturation of a heteropolyacid ammonium salt generated by an addition of ammonium radical is reduced; therefore, the size of particles (primary particles) formed by nuclear growth of the ammonium heteropolyacid salt is moderately increased. By drying the liquid A2 that contains primary particles having such a particle size in the below-described step (iii), a catalyst precursor having a preferred median diameter can be easily obtained. Further, the pores of the catalyst precursor are derived from voids between the primary particles generated in the liquid A2, and the primary particles tend to aggregate in a manner that the larger the size of the primary particles, the smaller is the amount of voids. Therefore, by drying the liquid A2 that contains the primary particles having the above-described particle size in the below-described step (iii), a catalyst precursor having a prescribed pore volume can be easily obtained. The value of v/M is more preferably 1.00 h⁻¹ or less, still more preferably 0.50 h⁻¹ or less.

The temperature of the liquid A1 at the time of adding the compound B is preferably 90 to 99° C. When the temperature of the liquid A1 is high at the time of adding the compound B, the degree of supersaturation of a heteropolyacid ammonium salt generated by an addition of ammonium radical is reduced; therefore, the size of the primary particles formed in the liquid A2 is increased, and the median diameter of the catalyst precursor generated when the liquid A2 is dried in the below-described step (iii) is increased as well. By controlling the temperature of the liquid A1 at the time of adding the compound B to be in the above-described range, a catalyst precursor having a preferred median diameter can be easily obtained. Further, the pores of the catalyst precursor are derived from voids between the primary particles generated in the liquid A2, and the primary particles tend to aggregate in a manner that the larger the size of the primary particles, the smaller is the amount of voids. Therefore, by drying the liquid A2 that contains the primary particles having the above-described particle size in the below-described step (iii), a catalyst precursor having a prescribed pore volume can be easily obtained. The lower limit of the temperature of the liquid A1 is more preferably 92° C. or higher, while the upper limit of the temperature of the liquid A1 is more preferably 98° C. or lower.

When adding the compound B, it is preferred to add the compound B while stirring the liquid A1 at a rotation speed of 70 to 140 rpm. When the rotation speed is high, the liquid A1 is sufficiently stirred, so that the degree of supersaturation of a heteropolyacid ammonium salt generated by an addition of ammonium radical is reduced. As a result, the size of the primary particles formed in the liquid A2 is increased, and the median diameter of the catalyst precursor generated when the liquid A2 is dried in the below-described step (iii) is increased as well. Further, the pores of the catalyst precursor are derived from voids between the primary particles generated in the liquid A2, and the primary particles tend to aggregate in a manner that the larger the size of the primary particles, the smaller is the amount of voids; therefore, the pore volume of the catalyst precursor is reduced. By stirring the liquid A1 at a rotation speed in the above-described range during the addition of the compound B, a catalyst precursor having a prescribed pore volume and a preferred median diameter can be easily obtained. The lower limit of the rotation speed is more preferably 100 rpm or more, while the upper limit of the rotation speed is more preferably 130 rpm or less. The liquid A1 can be stirred using a stirring device, such as a rotary blade stirrer.

### (Physical Properties of Liquid A2)

The resulting liquid A2 has a pH of 3 or lower, it is preferred that a lower limit thereof be 2 or higher, while the upper limit thereof be 2.8 or lower. When the liquid A2 has a pH of 3 or lower, a Keggin-type heteropolyacid suitable for the production of an α,β-unsaturated carboxylic acid is stably generated. As a method of controlling the pH of the liquid A2 to be 3 or lower, for example, in the preparation of the liquid A1, the pH of the liquid A1 may be adjusted by using molybdenum trioxide as a molybdenum raw material, or by appropriately selecting a raw material compound and thereby adjusting the content of nitrate ions or oxalate ions; or, in the preparation of the liquid A2, the content of ammonium ions or cesium ions may be adjusted.

The resulting liquid A2 has a solid concentration of preferably 30% by mass or lower, more preferably 22 to 27% by mass. By this, when the liquid A2 is dried in the below-described step (iii), droplets moderately shrink due to the evaporation of the solvent, so that a catalyst precursor having a preferred median diameter can be easily obtained. Further, when the liquid A2 is dried in the step (iii), those parts where the solvent evaporates from droplets become pores of the resulting catalyst precursor. When the solid concentration of the liquid A2 is in the above-described range, the liquid A2 contains the solvent in a moderate amount, so that a catalyst precursor having a prescribed pore volume can be easily obtained. The lower limit of the solid concentration of the liquid A2 is more preferably 23% by mass or higher, while the upper limit of the solid concentration of the liquid A2 is more preferably 26.5% by mass or lower.

The solid concentration of the liquid A2 can be adjusted by, for example, changing the weight ratio of the raw material compounds and the solvent to be used, and the added amount of the compound B and that of a G element-containing compound. When the added amount of the compound B and that of the G element-containing compound are increased, the precipitation of the resulting heteropolyacid salt is facilitated, the solid concentration is thereby increased. It is noted here that the solid concentration of the liquid A2 is defined as a value obtained by separating the liquid A2 into a solution and solids by centrifugation, measuring the mass thereof, and performing the calculation using the following Formula (III):

$$Solid concentration ( \% ) = Solid mass ( g ) / Total mass ( g ) of liquid A 2 \times 100 \qquad ( III )$$

In the liquid A2, when a ratio of a total mass of dissolved molybdenum element, phosphorus element, and vanadium element with respect to a total mass of molybdenum element, phosphorus element, and vanadium element is defined as R, R is preferably 5 to 25% by mass, more preferably 5 to 20% by mass. By this, when the liquid A2 is dried in the below-described step (iii), the dissolved components are precipitated in the voids between the primary particles contained in the liquid A2 and thereby moderately fill the voids, so that a catalyst precursor having a prescribed pore volume can be easily obtained. The lower limit of R is more preferably 6% by mass or more, while the upper limit of R is more preferably 15% by mass or less.

As a method of adjusting R, for example, the added amount of the compound B and that of the G element-containing compound may be changed, or the temperature of the liquid A1 at the time of adding the compound B may be changed. By increasing the added amount of the compound B and that of the G element-containing compound, the amount of the resulting heteropolyacid salt is increased, and R is thus reduced. Meanwhile, by raising the temperature of the liquid A1 at the time of adding the compound B, the solubility of the resulting heteropolyacid ammonium salt is increased, and R is thus increased. It is noted here that R is defined as a value calculated by the following Formula (IV) from the results of separating the liquid A2 into a solution and solids by centrifugation, measuring the mass thereof, and analyzing the thus obtained solution and solids by ICP emission spectrometry.

$$R ( \% by mass ) = \frac{{m}_{1} ( {M}_{1 Mo} + {M}_{1 P} + {M}_{1 V} )}{{m}_{1} ( {M}_{1 Mo} + {M}_{1 P} + {M}_{1 V} ) + {m}_{2} ( {M}_{2 Mo} + {M}_{2 P} + {M}_{2 V} )} \times 100 \qquad ( IV )$$

In Formula (IV), m₁ represents the weight (g) of the solution; m₂ represents the weight (g) of the solids; M_{1Mo} represents the molybdenum concentration (% by mass) in the solution; M_{1P} represents the phosphorus concentration (% by mass) in the solution; M_{1V} represents the vanadium concentration (% by mass) in the solution; M_{2Mo} represents the molybdenum ratio (% by mass) in the solids; M_{2P} represents the phosphorus ratio (% by mass) in the solids; and M_{2V} represents the vanadium ratio (% by mass) in the solids.

<Step (iii)>

In the step (iii), the liquid A2 obtained in the above-described step (ii) is dried to obtain dry particles as a catalyst precursor. Examples of a drying method include spray drying and drum drying, and spray drying is preferred. The spray drying can be performed using a spray dryer. The drum drying can be performed using a drum dryer.

In the spray dryer, a method of contact between sprayed droplets and a hot air may be any of a co-current method, a counter-current method, and a co/counter-current (mixed flow) method.

The spray dryer preferably has an inlet temperature of 100 to 500° C. The lower limit of the inlet temperature is more preferably 200° C. or higher, still more preferably 220° C. or higher, particularly preferably 240° C. or higher. Meanwhile, the upper limit of the inlet temperature is more preferably 400° C. or lower, still more preferably 370° C. or lower. The spray dryer has an outlet temperature of preferably 100 to 200° C., more preferably 105 to 200° C. For example, by increasing the inlet temperature and the outlet temperature of the spray dryer, the solvent is rapidly evaporated; therefore, shrinkage of droplets during the evaporation of the solvent is unlikely to occur, so that the resulting catalyst precursor tends to have a large pore volume and a large median diameter. By setting the inlet temperature and the outlet temperature of the spray dryer in the above-described respective ranges, a catalyst precursor having a prescribed pore volume and a preferred median diameter can be easily obtained.

The spray drying is preferably performed such that the resulting catalyst precursor has a water content of 0.1 to 4.5% by mass.

A catalyst precursor can be produced in the above-described manner. The catalyst precursor may have a catalytic activity; however, from the standpoint of the α,β-unsaturated carboxylic acid yield, it is preferred to use a catalyst produced by performing the below-described molding and/or calcination.

### [Method of Producing Catalyst]

The method of producing a catalyst according to the present embodiment is a method of producing a catalyst used for the production of an α,β-unsaturated carboxylic acid by oxidation of an α,β-unsaturated aldehyde, and includes molding and/or calcinating the catalyst precursor according to the present embodiment. The term “molding and/or calcinating” indicates that only molding or calcination may be performed, or both molding and calcination may be performed.

### <Molding of Catalyst Precursor>

A method of molding the catalyst precursor is not particularly limited, and any known dry or wet molding method, examples of which include tablet molding, press molding, compression molding, extrusion molding, and granulation molding, can be applied. A shape of the resulting molded article is not particularly limited, and may be, for example, a columnar shape, a ring shape, or a spherical shape. At the time of the molding, it is preferred to mold only the catalyst precursor without adding a carrier or the like thereto; however, if necessary, a known additive, such as graphite or talc, may be added as well. In the case of using a carrier, the carrier is not particularly limited, and one preferred example thereof is silica. The molding may be performed after the below-described calcination.

### <Calcination of Catalyst Precursor>

The calcination can be performed in a stream of at least one of an oxygen-containing gas such as air or an inert gas, and the calcination is preferably performed in a stream of an oxygen-containing gas such as air. The “inert gas” refers to a gas that does not cause a reduction in the catalyst activity, and examples thereof include nitrogen, carbon dioxide, helium, and argon. These inert gases may be used singly, or in combination of two or more thereof.

A calcination method is not particularly limited to the use of, for example, a fluidized bed, a rotary kiln, a muffle furnace, or a tunnel firing furnace, and an appropriate method can be selected taking into consideration the performance, the mechanical strength, the moldability, the production efficiency, and the like of the catalyst to be eventually obtained.

The calcination temperature (highest temperature during calcination) is preferably 200 to 700° C., and the lower limit thereof is more preferably 320° C. or higher, while the upper limit thereof is more preferably 450° C. or lower.

### [Molded Catalyst Product]

The molded catalyst product according to the present embodiment is used for the production of an α,β-unsaturated carboxylic acid by oxidation of an α,β-unsaturated aldehyde, and contains a catalyst component that contains phosphorus, molybdenum, and vanadium. The molded catalyst product has a pore volume of 0.01 to 0.40 mL/g and, in a pore distribution curve of the molded catalyst product, when the height of a highest peak (peak A) and that of a second highest peak (peak B), apexes of which exist in a pore diameter range of 0.05 to 10 μm, are defined as IA and IB, respectively, a ratio IB/IA is 0.160 to 0.420.

Studies have been conducted on each of the pore volume and the pore distribution of a molded catalyst product for the production of an α,β-unsaturated carboxylic acid. However, it is difficult to produce a molded catalyst product by controlling these properties independently, and no study has been conducted on the control of both of the pore volume and the pore distribution of a molded catalyst product. In addition, conventionally, there is a problem that, while an increase in the pore volume of a molded catalyst product improves the α,β-unsaturated carboxylic acid yield, it reduces the weight of the molded catalyst product that can be packed into a reactor of a certain volume. Therefore, the effect of improving the α,β-unsaturated carboxylic acid yield by an increase in the pore volume of the molded catalyst product cannot be obtained sufficiently.

In the present invention, as a result of studies conducted on the pore distribution of a molded catalyst product, it was discovered that the above-described problem can be solved by controlling the ratio IB/IA to be in a prescribed range. It was also discovered that an α,β-unsaturated carboxylic acid can be produced with a particularly high yield by controlling the pore volume of the molded catalyst product to be in a prescribed range.

### <Composition of Catalyst Component>

The molded catalyst product according to the present embodiment contains a catalyst component that contains phosphorus, molybdenum, and vanadium. From the standpoint of improving the α,β-unsaturated carboxylic acid yield, preferred modes of the catalyst component in terms of the ratio of the number of phosphorus atoms with respect to the number of molybdenum atoms and the ratio of the number of vanadium atoms with respect to the number of molybdenum atoms are the same as those of the catalyst precursor according to the present embodiment.

From the standpoint of improving the α,β-unsaturated carboxylic acid yield, the catalyst component particularly preferably has a composition represented by the above-described Formula (I) in the same manner as the catalyst precursor according to the present embodiment. The catalyst component may also contain a small amount of an element that is not included in the above-described Formula (I). With regard to the composition represented by the above-described Formula (I), preferred modes of the catalyst component are the same as those of the catalyst precursor according to the present embodiment.

### <Pore Volume and Pore Distribution of Molded Catalyst Product>

The molded catalyst product according to the present embodiment has a pore volume of 0.01 to 0.40 mL/g. When the pore volume is 0.01 mL/g or more, an α,β-unsaturated aldehyde used as a raw material can be sufficiently diffused to adequately interact with surface active sites; therefore, the reaction rate is improved. Further, since the resulting α,β-unsaturated carboxylic acid is also sufficiently diffused, a reaction in which the α,β-unsaturated carboxylic acid is converted into a by-product through sequential oxidation can be inhibited. When the pore volume is 0.4 mL/g or less, the molded catalyst product has a sufficiently high density, and this leads to an increase in the weight of the molded catalyst product that can be packed into a reactor of a certain volume; therefore, the α,β-unsaturated carboxylic acid yield is improved. The lower limit of the pore volume is preferably 0.10 mL/g or more, more preferably 0.20 mL/g or more. The upper limit of the pore volume is preferably 0.35 mL/g or less, more preferably 0.30 mL/g or less.

Examples of a method of obtaining a molded catalyst product having a pore volume in the above-described range include a method of producing a molded catalyst product by the below-described production method in which the addition rate v (mol/h) of the compound B, the temperature of the liquid A1, and the stirring rotation speed of the liquid A1 are adjusted in the step (ii), the physical properties of the liquid A2 are adjusted, the drying conditions are adjusted in the step (iii), or the extrusion molding conditions are adjusted in the step (iv).

In the pore distribution curve of the molded catalyst product according to the present embodiment, when the height of a highest peak (peak A) and that of a second highest peak (peak B), apexes of which exist in a pore diameter range of 0.05 to 10 μm, are defined as IA and IB, respectively, a ratio IB/IA is 0.160 to 0.420. A pore distribution that satisfies this condition is particularly effective for the diffusion of an α,β-unsaturated aldehyde used as a raw material and the resulting α,β-unsaturated carboxylic acid and, regardless of the pore volume, the α,β-unsaturated aldehyde and the α,β-unsaturated carboxylic acid can be sufficiently diffused. It is presumed that, as a result, an improvement in the α,β-unsaturated carboxylic acid yield by sufficient diffusion of the α,β-unsaturated aldehyde and the α,β-unsaturated carboxylic acid, as well as an improvement in the α,β-unsaturated carboxylic acid yield by an increase in the weight of the molded catalyst product that can be packed into a reactor of a certain volume can be achieved at the same time, and a large effect can be obtained. The lower limit of the ratio IB/IA is preferably 0.200 or higher, while the upper limit of the ratio IB/IA is preferably 0.400 or lower.

In the pore distribution curve, the number of peaks existing in a pore diameter range of 0.05 to 10 μm is preferably 4 or less, more preferably 3 or less, still more preferably 2.

The apexes of the peaks A and B exist in a range of preferably 0.05 μm or more, more preferably 0.08 μm or more, still more preferably 0.10 μm or more. Further, the apexes of the peaks A and B exist in a range of preferably 10 μm or less, more preferably 8 μm or less, still more preferably 6 μm or less. When the peaks A and B are in this range, the pore distribution is suitable for the diffusion of the α,β-unsaturated aldehyde and the α,β-unsaturated carboxylic acid, so that the α,β-unsaturated carboxylic acid yield is improved.

In the present invention, the pore volume and the pore distribution of the molded catalyst product are measured by a mercury intrusion method. The pore volume and the pore distribution of the molded catalyst product can be measured using, for example, a pore distribution analyzer such as AutoPore IV-9500 (product name, manufactured by Micromeritics Instrument Corporation). In the present invention, the “pore size distribution curve” refers to a log differential pore volume distribution curve. The “log differential pore volume distribution” is a graph obtained by dividing the differential pore volume by the common logarithmic difference value of the pore diameter, and plotting the thus obtained values against the average pore diameter of each interval. Further, in the present invention, the apex of a peak refers to a position at which the first derivative value of the pore size distribution curve is 0 and the second derivative value is negative. Moreover, the height of a peak refers to the distance from a horizontal line having an ordinate value of 0 to the apex of the peak.

### <Specific Surface Area of Molded Catalyst Product>

The molded catalyst product according to the present embodiment preferably has a specific surface area of 1 to 10 m²/g. When the specific surface area is 1 m²/g or more, the number of active sites that can interact with the α,β-unsaturated aldehyde used as a raw material is sufficiently large, so that the α,β-unsaturated carboxylic acid yield is improved. Meanwhile, when the specific surface area is 10 m²/g or less, the below-described pore volume can be easily controlled to be in a prescribed range. The lower limit of the specific surface area is more preferably 1.5 m²/g or more, still more preferably 2 m²/g or more. The upper limit of the specific surface area is more preferably 8 m²/g or less, still more preferably 6 m²/g or less, particularly preferably 4 m²/g or less.

In the present invention, the specific surface area of the molded catalyst product is a value determined by a nitrogen adsorption method. The specific surface area of the molded catalyst product can be determined by a 5-point BET method using, for example, TriStar 3000 (product name, manufactured by Micromeritics Instrument Corporation).

Examples of a method of obtaining a molded catalyst product having a specific surface area in the above-described range include a method of producing a molded catalyst product by the below-described production method in which the addition rate v (mol/h) of the compound B, the temperature of the liquid A1, and the stirring rotation speed of the liquid A1 are adjusted in the step (ii), or the physical properties of the liquid A2 are adjusted.

### <Shape of Molded Catalyst Product>

A shape of the molded catalyst product is not particularly limited, and may be any shape, such as a spherical shape, a cylindrical shape, a ring shape, a star shape, or a granular shape obtained by post-molding pulverization and classification. Among these shapes, from the standpoint of mechanical strength, the molded catalyst product preferably has a spherical shape, a cylindrical shape, or a ring shape. The size of the molded article is also not particularly limited; however, for example, in the case of a spherical shape, the diameter of the sphere is preferably 0.1 to 10 mm. The lower limit of the diameter of the sphere is more preferably 0.5 mm or more, still more preferably 1 mm or more, particularly preferably 3 mm or more. Meanwhile, the upper limit of the diameter of the sphere is more preferably 8 mm or less, still more preferably 6 mm or less. In the case of a ring shape or a cylindrical shape, the diameter of the bottom circle and the height of the ring or the cylinder are both preferably 0.1 to 10 mm. The lower limit of the diameter and the height is more preferably 0.5 mm or more, still more preferably 1 mm or more, particularly preferably 3 mm or more. Meanwhile, the upper limit of the diameter and the height is more preferably 8 mm or less, still more preferably 6 mm or less. In the case of other shapes, the length between two points farthest away from each other in the three-dimensional shape of the molded catalyst product is preferably 0.1 to 10 mm. The lower limit of the length between the two points is more preferably 0.5 mm or more, still more preferably 1 mm or more, particularly preferably 3 mm or more. Meanwhile, the upper limit of the length between the two points is more preferably 8 mm or less, still more preferably 6 mm or less. By this, the yield of a desired product and the continuous operational period of the molded catalyst product are improved.

It is noted here that the molded catalyst product according to the present embodiment is preferably an extrusion-molded article.

### [Method of Producing Molded Catalyst Product]

The molded catalyst product according to the present embodiment can be produced in accordance with any known molded catalyst product production method, as long as the molded catalyst product contains a catalyst component containing phosphorus, molybdenum, and vanadium, and has a pore volume of 0.01 to 0.40 mL/g and a ratio IB/IA of 0.160 to 0.420 in a pore distribution curve where the height of a highest peak (peak A) and that of a second highest peak (peak B), apexes of which exist in a pore diameter range of 0.05 to 10 μm, are defined as IA and IB, respectively; however, the molded catalyst product according to the present embodiment is preferably produced by the method of producing a molded catalyst product according to the present embodiment, which method includes the above-described steps (i) to (iii) and the following step (iv):
(iv) the step of extrusion-molding the above-described dry particles to obtain a molded catalyst product.

The method of producing a molded catalyst product according to the present embodiment may further include the below-described calcination step.

In the method of producing a molded catalyst product according to the present embodiment, preferred modes of the steps (i) to (iii) are the same as those of the method of producing a catalyst precursor according to the present embodiment.

Satisfaction of Formula (II) in the step (ii) means that the ammonium radical addition rate is sufficiently low. By this, the degree of supersaturation of a heteropolyacid ammonium salt generated by an addition of ammonium radical is reduced; therefore, the size of particles (primary particles) formed by nuclear growth of the ammonium heteropolyacid salt is increased, so that the strength of the dry particles generated when the liquid A2 is dried in the step (iii) is improved.

Further, by extrusion-molding the dry particles having a high strength in the below-described step (iv), the shape of the dry particles is maintained even after the molding. As a result, two kinds of pores, which are pores inside the dry particles that are derived from voids between the primary particles and pores derived from voids between the dry particles, are formed in the resulting molded catalyst product. Usually, the pores derived from voids between the dry particles give the peak A, while the pores inside the dry particles that are derived from voids between the primary particles give the peak B. By adjusting the extrusion molding conditions of the below-described step (iv), the ratio of the pores inside the dry particles that are derived from voids between the primary particles and the pores derived from voids between the dry particles can be adjusted, so that a molded catalyst product having a ratio IB/IA in a prescribed range can be easily obtained.

An increase in the size of the primary particles generated in the liquid A2 also leads to an increase in the size of the dry particles. In this case, the voids between the primary particles and the voids between the dry particles become pores when the dry particles are extrusion-molded in the below-described step (iv); therefore, the peak pore diameter in the pore distribution is increased. By adding an ammonium radical such that the above-described Formula (II) is satisfied, a molded catalyst product having the apexes of the peaks A and B in a preferred range can be easily obtained.

In Formula (II), the value of v/M is more preferably 1.00 h⁻¹ or less, still more preferably 0.50 h⁻¹ or less.

The temperature of the liquid A1 at the time of adding the compound B is preferably 90 to 99° C. When the temperature of the liquid A1 is low at the time of adding the compound B, the degree of supersaturation of a heteropolyacid ammonium salt generated by an addition of ammonium radical is increased; therefore, the size of the primary particles formed in the liquid A2 is reduced, and the specific surface area of the molded catalyst product obtained in the below-described step (iv) is increased. By controlling the temperature of the liquid A1 at the time of adding the compound B to be in the above-described range, a molded catalyst product having a preferred specific surface area can be easily obtained.

A reduction in the size of the primary particles generated in the liquid A2 also leads to a reduction in the size of the dry particles, and the voids between the primary particles and the voids between the dry particles become pores when the dry particles are molded; therefore, the peak pore diameter in the pore distribution is reduced. By controlling the temperature of the liquid A1 at the time of adding the compound B to be 90 to 99° C., a molded catalyst product having the apexes of the peaks A and B in a preferred range can be easily obtained. The lower limit of the temperature of the liquid A1 is more preferably 92° C. or higher, while the upper limit of the temperature of the liquid A1 is more preferably 98° C. or lower.

When adding the compound B, it is preferred to add the compound B while stirring the liquid A1 at a rotation speed of 70 to 140 rpm. The liquid A1 can be stirred using a stirring device, such as a rotary blade stirrer. By stirring the liquid A1 at a rotation speed of 70 rpm or more, the liquid A1 is sufficiently stirred; therefore, the degree of supersaturation of a heteropolyacid ammonium salt generated by an addition of ammonium radical is reduced. As a result, for the same reason as described above, the strength of the dry particles generated when the liquid A2 is dried in the step (iii) is improved, so that a molded catalyst product having a ratio IB/IA in a prescribed range can be easily obtained. Further, a molded catalyst product having the apexes of the peaks A and B in a preferred range can be easily obtained.

By stirring the liquid A1 at a rotation speed of 140 rpm or less, the degree of supersaturation of a heteropolyacid ammonium salt generated by an addition of ammonium radical is increased; therefore, the size of the primary particles formed in the liquid A2 is reduced. As a result, in the below-described step (iv), a molded catalyst product having a preferred specific surface area can be easily obtained.

A lower limit of the rotation speed is more preferably 100 rpm or more, while an upper limit of the rotation speed is more preferably 130 rpm or less.

The resulting liquid A2 has a solid concentration of preferably 30% by mass or lower, more preferably 22 to 27% by mass. When the liquid A2 is dried in the step (iii), those parts where the solvent evaporates from droplets become pores of the resulting dry particles. When the solid concentration of the liquid A2 is in the above-described range, the liquid A2 contains the solvent component in a moderate amount, so that dry particles having an appropriate pore volume can be easily obtained. By molding the dry particles, a molded catalyst product having a prescribed pore volume can be easily obtained. The lower limit of the solid concentration is more preferably 23% by mass or higher, while the upper limit of the solid concentration is more preferably 26.5% by mass or lower.

In the liquid A2, when a ratio of a total mass of dissolved molybdenum element, phosphorus element, and vanadium element with respect to a total mass of molybdenum element, phosphorus element, and vanadium element is defined as R, R is preferably 5 to 25% by mass, more preferably 5 to 20% by mass. By this, when the liquid A2 is dried in the step (iii), the dissolved components are precipitated in the voids between the primary particles contained in the liquid A2 and thereby moderately fill the voids, so that dry particles having an appropriate pore volume can be easily obtained. Further, by molding the dry particles, a molded catalyst product having a prescribed pore volume can be easily obtained.

In addition, since the strength of the dry particles is improved as a result of the filling of the voids therein, the shape of the dry particles is maintained even after the dry particles are extrusion-molded in the below-described step (iv). As a result, two kinds of pores, which are pores inside the dry particles that are derived from the voids between the primary particles and pores derived from the voids between the dry particles, are formed in the resulting molded catalyst product. By adjusting the extrusion molding conditions of the below-described step (iv), the ratio of the pores inside the dry particles that are derived from the voids between the primary particles and the pores derived from the voids between the dry particles can be adjusted, so that a molded catalyst product having a ratio IB/IA in a prescribed range can be easily obtained.

The lower limit of R is more preferably 6% by mass or more, while the upper limit of R is more preferably 15% by mass or less.

In the step (iii), when the drying method is spray drying using a spray dryer, the spray dryer preferably has an inlet temperature of 100 to 500° C. The lower limit of the inlet temperature is more preferably 200° C. or higher, still more preferably 220° C. or higher, particularly preferably 240° C. or higher. Meanwhile, the upper limit of the inlet temperature is more preferably 400° C. or lower, still more preferably 370° C. or lower. The spray dryer has an outlet temperature of preferably 100 to 200° C., more preferably 105 to 200° C. For example, by lowering the inlet temperature and the outlet temperature of the spray dryer, the solvent is slowly evaporated; therefore, shrinkage of droplets during the evaporation of the solvent is likely to occur, and this tends to reduce the pore volume of the resulting dry particles as well as the pore volume of the resulting molded catalyst product. By setting the inlet temperature and the outlet temperature of the spray dryer in the above-described respective ranges, dry particles having a prescribed pore volume can be easily obtained.

Further, by setting the inlet temperature and the outlet temperature of the spray dryer at the above-described respective temperatures or more, the strength of the dry particles is improved; therefore, the shape of the dry particles is maintained even after the dry particles are extrusion-molded in the below-described step (iv). As a result, two kinds of pores, which are pores inside the dry particles that are derived from the voids between the primary particles and pores derived from the voids between the dry particles, are formed in the resulting molded catalyst product. By adjusting the extrusion molding conditions of the below-described step (iv), the ratio of the pores inside the dry particles that are derived from the voids between the primary particles and the pores derived from the voids between the dry particles can be adjusted, so that a molded catalyst product having a ratio IB/IA in a prescribed range can be easily obtained.

The spray drying is preferably performed such that the resulting dry particles have a water content of 0.1 to 4.5% by mass.

<Step (iv)>

In the step (iv), the dry particles obtained in the above-described step (iii) are extrusion-molded to obtain a molded catalyst product. It is noted here that the extrusion molding may be performed after the below-described calcination step.

By extrusion-molding the dry particles, a molded catalyst product having a prescribed pore volume and a prescribed pore distribution can be easily obtained. In the case of using a carrier, the carrier is not particularly limited, and one preferred example thereof is silica.

The extrusion molding may be performed after pulverizing the dry particles if necessary. Further, depending on the properties of the dry particles, the dry particles may be mixed with additives to obtain a kneaded product, and this kneaded product may be extrusion-molded using an extruder. It is preferred to obtain a kneaded product by mixing the dry particles with a liquid and additives and then extrusion-mold the kneaded product using an extruder, and it is more preferred to obtain a kneaded product by mixing the dry particles with a liquid and a binder and then extrusion-mold the kneaded product using an extruder.

(Liquid Mixed with Dry Particles)

The liquid to be mixed with the dry particles is not particularly limited as long as it has a function of wetting the dry particles, and examples thereof include water and alcohols having 1 to 4 carbon atoms, such as methanol, ethanol, propanol, and butanol. Thereamong, from the standpoint of the ease of forming pores effective for the oxidation reaction of an α,β-unsaturated aldehyde without disintegration of the dry particles, ethanol and propanol are preferred. These liquids may be used singly, or in combination of two or more kinds thereof.

The amount of the liquid to be used is selected as appropriate in accordance with the type and the size of the dry particles, the type of the liquid, and the like; however, it is preferably 15 to 60 parts by mass with respect to 100 parts by mass of the dry particles. When the amount of the liquid is increased, disintegration of the dry particles during the extrusion molding is made unlikely to occur, and the amount of the pores derived from the voids between the dry particles is increased; therefore, the ratio IB/IA tends to be reduced. By controlling the amount of the liquid to be 15 parts by mass or more with respect to 100 parts by mass of the dry particles, a molded catalyst product having a ratio IB/IA in a prescribed range can be easily obtained. Further, by controlling the amount of the liquid to be 60 parts by mass or less, a molded catalyst product having a low total pore volume and a high strength can be easily obtained. The lower limit of the amount of the liquid to be used with respect to 100 parts by mass of the dry particles is more preferably 16 parts by mass or more. Meanwhile, the upper limit of the same is more preferably 50 parts by mass or less, still more preferably 45 parts by mass or less, particularly preferably 35 parts by mass or less.

(Additives Mixed with Dry Particles)

The additives to be mixed with the dry particles are not particularly limited, and known additives, such as graphite, talc, organic binders, and inorganic binders, can be used. Thereamong, from the standpoint of improving the moldability in the extrusion molding, it is preferred to mix an organic binder. Examples of the organic binder include: polymer compounds, such as polyvinyl alcohol; α-glucan derivatives; and β-glucan derivatives. These organic binders may be used singly, or in combination of two or more kinds thereof.

The α-glucan derivatives are polysaccharides composed of glucose, in which glucose is bound in an α-type structure. Examples of the α-glucan derivatives include derivatives of α1-4 glucan, α1-6 glucan, α1-4/1-6 glucan, and the like. Examples of these α-glucan derivatives include amylose, glycogen, amylopectin, pullulan, dextrin, and cyclodextrin. These α-glucan derivatives may be used singly, or in combination of two or more kinds thereof.

The β-glucan derivatives are polysaccharides composed of glucose, in which glucose is bound in a β-type structure. Examples of the β-glucan derivatives include derivatives of 01-4 glucan, β1-3 glucan, β1-6 glucan, 01-3/1-6 glucan, and the like. Examples of these β-glucan derivatives include cellulose derivatives, such as methylcellulose, ethylcellulose, carboxymethylcellulose, sodium carboxymethylcellulose, hydroxyethylcellulose, hydroxypropylcellulose, hydroxypropylmethylcellulose, hydroxyethylmethylcellulose, hydroxybutylmethylcellulose, and ethylhydroxyethylcellulose, as well as curdlan, laminaran, paramylon, callose, pachyman, and scleroglucan. These β-glucan derivatives may be used singly, or in combination of two or more kinds thereof.

The organic binder may be used in an unpurified state, or may be purified before use. However, when a metal or an ignition residue is contained as an impurity, the catalyst performance may be deteriorated; therefore, the smaller the content thereof, the more preferred it is.

The amount of the organic binder to be used is selected as appropriate in accordance with the type and the size of the dry particles, the type of the liquid, and the like; however, it is preferably 0.05 to 15 parts by mass with respect to 100 parts by mass of the dry particles, and the lower limit thereof is more preferably 0.1 parts by mass or more, while the upper limit thereof is more preferably 10 parts by mass or less. When the amount of the organic binder used is 0.05 parts by mass or more, the moldability in the extrusion molding is improved. Further, those parts where the organic binder is removed by performing the below-described calcination step become pores, so that a molded catalyst product having a prescribed pore volume can be easily obtained. Meanwhile, when the amount of the organic binder used is 15 parts by mass or less, the organic binder can be easily removed by performing the below-described calcination step; therefore, an adverse effect on the catalyst performance can be inhibited.

(Mixing of Dry Particles with Liquid and Additives)

The mixing of the dry particles with the liquid and the additives is preferably done by kneading these materials using, for example, a batch-type kneader equipped with a dual-arm stirring blade, or a continuous-type kneader such as an axial-rotation reciprocating-type kneader or a self-cleaning-type kneader. As a kneader, a batch-type kneader is preferred from the standpoint of allowing verification of the state of kneading. An end point of the kneading is defined as the time when the materials have been mixed to an extrusion-moldable state, and the end point is determined visually or by touch.

### (Extrusion Molding)

In the extrusion molding, the dry particles or a mixture thereof is placed in a mold, extruded with a pressure, and thereby molded into a certain shape to obtain an extrusion-molded article. For the extrusion molding, for example, a screw-type extrusion molding machine or a plunger-type extrusion molding machine can be used, and it is preferred to use a plunger-type extrusion molding machine.

The extrusion pressure is preferably 0.1 to 30 MPa. By controlling the extrusion pressure to be 0.1 MPa or higher, a molded catalyst product having a prescribed pore volume and a high mechanical strength can be stably obtained. Further, when the extrusion pressure is low, destruction of the dry particles constituting the molded catalyst product is inhibited, and the amount of the pores derived from the voids between the dry particles is increased; therefore, the ratio IB/IA tends to be reduced. By controlling the extrusion pressure to be 30 MPa or lower, a molded catalyst product having a ratio IB/IA in a prescribed range can be easily obtained. The lower limit of the extrusion pressure is more preferably 0.5 MPa or higher, still more preferably 1 MPa or higher, particularly preferably 2 MPa or higher. Meanwhile, the upper limit of the extrusion pressure is more preferably 20 MPa or lower, still more preferably 15 MPa or lower.

The resulting molded catalyst product may be cut to a desired size if necessary. A cutting method can be selected from any known methods, and examples thereof include a method using a rotating cutting blade, and a method using a reciprocating cutting blade.

When the dry particles and the liquid are mixed and extrusion-molded, if necessary, the resulting molded catalyst product is preferably maintained at a temperature of 10 to 200° C. to remove the liquid contained in the molded catalyst product. By setting the temperature to 10° C. or higher, the liquid component can be sufficiently removed. Further, by setting the temperature to 200° C. or lower, deterioration of the molded catalyst product can be prevented. The lower limit of the retention temperature is more preferably 20° C. or higher. Meanwhile, the upper limit of the retention temperature is more preferably 180° C. or lower, still more preferably 150° C. or lower, particularly preferably 120° C. or lower. It is noted here that the liquid may be removed by drying the molded catalyst product using any commonly known dryer. The operating conditions of the dryer are not particularly limited and, for example, the molded catalyst product can be maintained in an air atmosphere or a nitrogen atmosphere.

The molded catalyst product obtained in the step (iv) exhibits catalytic performance and can be used for the production of an α,β-unsaturated carboxylic acid; however, the molded catalyst product is preferably further subjected to the below-described calcination since, as a result thereof, a molded catalyst product having prescribed specific surface area, pore volume, and pore distribution curve can be easily obtained, and the α,β-unsaturated carboxylic acid yield is improved. In the present invention, a molded catalyst product obtained after the calcination is also generally included in the term “molded catalyst product”.

### <Calcination Step>

In the calcination step, the dry particles obtained in the step (iii) or the molded catalyst product obtained in the step (iv) is calcined as required.

The calcination can be performed in a stream of at least one of an oxygen-containing gas such as air or an inert gas, and the calcination is preferably performed in a stream of an oxygen-containing gas such as air. The “inert gas” refers to a gas that does not cause a reduction in the catalyst activity, and examples thereof include nitrogen, carbon dioxide, helium, and argon. These inert gases may be used singly, or in combination of two or more thereof.

A calcination method is not particularly limited to the use of, for example, a fluidized bed, a rotary kiln, a muffle furnace, or a tunnel firing furnace, and an appropriate method can be selected taking into consideration the performance, the mechanical strength, the moldability, the production efficiency, and the like of the catalyst to be eventually obtained.

The calcination temperature (highest temperature during calcination) is preferably 200 to 700° C., and the lower limit thereof is more preferably 320° C. or higher, while the upper limit thereof is more preferably 450° C. or lower.

A molded catalyst product can be produced in the above-described manner.

### [Method of Producing α,β-Unsaturated Carboxylic Acid]

In the method of producing an α,β-unsaturated carboxylic acid according to the present embodiment, an α,β-unsaturated aldehyde is oxidized using a catalyst obtained by molding and/or calcinating the catalyst precursor according to the present embodiment, or the molded catalyst product according to the present embodiment. Alternatively, in the method of producing an α,β-unsaturated carboxylic acid according to the present embodiment, an α,β-unsaturated aldehyde is oxidized using a catalyst that is obtained by molding and/or calcinating a catalyst precursor produced by the method of producing a catalyst precursor according to the present embodiment, or a molded catalyst product produced by the method of producing a molded catalyst product according to the present embodiment. By these methods, an unsaturated carboxylic acid can be produced with a high yield. From the standpoint of the product yield, the α,β-unsaturated aldehyde and the α,β-unsaturated carboxylic acid are preferably methacrolein and methacrylic acid, respectively.

The method of producing an α,β-unsaturated carboxylic acid according to the present embodiment can be carried out by bringing a catalyst or a molded catalyst product that is obtained by molding and/or calcinating the catalyst precursor according to the present embodiment into contact with an α,β-unsaturated aldehyde-containing raw material gas. A fixed-bed reactor can be used for this reaction.

The catalyst or the molded catalyst product are packed into the reactor, and the reaction can be performed by supplying the raw material gas into the reactor. The catalyst or the molded catalyst product may be provided in a single layer, or plural catalysts or molded catalyst products having different activities may each be packed in plural separate layers. Further, in order to control the activity, the catalyst or the molded catalyst product may be diluted with an inert carrier before being packed.

The concentration of the α,β-unsaturated aldehyde in the raw material gas is preferably 1 to 20% by volume, and the lower limit thereof is more preferably 3% by volume or higher, while the upper limit thereof is more preferably 10% by volume or lower. The α,β-unsaturated aldehyde, which is a raw material, may contain a small amount of impurities that do not substantially affect the reaction, such as lower saturated aldehydes.

An oxygen source of the raw material gas is not particularly limited; however, it is industrially advantageous to use air. If necessary, a gas obtained by mixing air or the like with pure oxygen can be used as well. A ratio of oxygen in the raw material gas is not particularly limited; however, it is preferably 0.4 to 4 mol with respect to 1 mol of the α,β-unsaturated aldehyde, and the lower limit thereof is more preferably 0.5 mol or higher, while the upper limit thereof is more preferably 3 mol or lower.

From the economic standpoint, the raw material gas may be diluted with an inert gas such as nitrogen or carbon dioxide. Further, water vapor may be added to the raw material gas. By performing the reaction in the presence of water vapor, an α,β-unsaturated carboxylic acid can be obtained with a higher yield. The concentration of water vapor in the raw material gas is preferably 0.1 to 50% by volume, and the lower limit thereof is more preferably 1% by volume or higher, while the upper limit thereof is more preferably 40% by volume.

A contact time between the raw material gas and the catalyst or the molded catalyst product for the production of an α,β-unsaturated carboxylic acid is preferably 0.5 to 15 seconds, and the lower limit thereof is more preferably 1.5 seconds or longer, while the upper limit thereof is more preferably 10 seconds or shorter. The reaction pressure is preferably 0 to 1 MPaG. It is noted here that “G” represents a gauge pressure, and 0 MPaG means that the reaction pressure is an atmospheric pressure. The reaction temperature is preferably 200 to 450° C., and the lower limit thereof is more preferably 250° C. or higher, while the upper limit thereof is more preferably 400° C. or lower.

### [Method of Producing α,β-Unsaturated Carboxylic Acid Ester]

In the method of producing an α,β-unsaturated carboxylic acid ester according to the present embodiment, an α,β-unsaturated carboxylic acid produced by the production method according to the present embodiment is esterified. In other words, the method of producing an α,β-unsaturated carboxylic acid ester according to the present embodiment includes: the step of producing an α,β-unsaturated carboxylic acid by the method according to the present embodiment; and the step of esterifying the α,β-unsaturated carboxylic acid.

An alcohol to be reacted with the α,β-unsaturated carboxylic acid is not particularly limited, and examples thereof include methanol, ethanol, n-propanol, isopropanol, n-butanol, and isobutanol. Examples of the resulting α,β-unsaturated carboxylic acid ester include methyl (meth)acrylate, ethyl (meth)acrylate, propyl (meth)acrylate, isopropyl (meth)acrylate, butyl (meth)acrylate, and isobutyl (meth)acrylate. The esterification reaction can be performed in the presence of an acidic catalyst, such as a sulfonic acid-type cation exchange resin. The temperature during the esterification reaction is preferably 50 to 200° C.

### EXAMPLES

The present invention will now be described concretely by way of Examples and Comparative Examples; however, the present invention is not limited to the below-described Examples. It is noted here that “part(s)” denotes “part(s) by mass”.

### (Composition Ratio of Catalyst Precursor and Composition Ratio of Catalyst Component)

The molar ratio of each component was determined by analyzing the components of a catalyst precursor or molded catalyst product dissolved in aqueous ammonia using an ICP emission spectrometer OPTIMA 8300 ICP-OES (product name, manufactured by PerkinElmer Co., Ltd.). Further, the molar ratio of ammonium radical was determined by analyzing the catalyst precursor or molded catalyst product by the Kjeldahl method.

### (Specific Surface Area of Molded Catalyst Product)

The specific surface area of a molded catalyst product was measured using a nitrogen adsorption measuring device TriStar 3000 (product name, manufactured by Micromeritics Instrument Corporation) for 1.5 g of the molded catalyst product pretreated at 200° C., and calculated by a five-point BET method.

### (Pore Volume of Catalyst Precursor)

The pore volume of a catalyst precursor was measured using a mercury-intrusion pore distribution analyzer AutoPore IV-9500 (product name, manufactured by Micromeritics Instrument Corporation) under the following conditions, and calculated from the volume of pores having a pore diameter of 0.5 μm or less.
Amount of measured sample: 1.5 g
Sample pretreatment: 12-hour heating at 90° C.
Cell capacity: 1.131 mL
Initial vacuuming: 5 minutes at 50 μm Hg
Equilibrium waiting time: 5 minutes
Measurement pressure range: 0.0038 to 207 MPa
Measurement pore diameter range: 0.006 to 329 μm

### (Pore Volume and Pore Distribution of Molded Catalyst Product)

The pore volume and the pore distribution of a molded catalyst product were measured using a mercury-intrusion pore distribution analyzer AutoPore IV-9500 (product name, manufactured by Micromeritics Instrument Corporation) under the following conditions.
Measurement sample: 10 particles of molded catalyst product
Cell capacity: 1.131 mL
Initial vacuuming: 5 minutes at 50 μm Hg
Equilibrium waiting time: 5 minutes
Measurement pressure range: 0.0038 to 207 MPa
Measurement pore diameter range: 0.006 to 329 μm

The physical property values of mercury used for the measurement using the mercury-intrusion pore distribution analyzer were as follows.
Mercury contact angle: 130°
Mercury surface tension: 485 dynes/cm
Mercury density: 13.5335 g/mL

### (Median Diameter of Catalyst Precursor)

The median diameter of a catalyst precursor was measured using a laser diffraction-type wet particle size distribution analyzer LA-700 (product name, manufactured by Horiba, Ltd.) under the following conditions.
Dispersion medium: ethanol
Refractive index setting: 1.8

### (Analysis of Keggin-Type Heteropolyacid in Catalyst Precursor)

The presence or absence of a Keggin-type heteropolyacid in a catalyst precursor was determined by an infrared absorption analysis using NICOLET 6700FT-IR (product name, manufactured by Thermo Electron Co., Ltd.). The FT-IR measurement was performed by a transmission method. First, KBr was molded into pellets using a tablet molding machine, and background measurement was performed. Next, a catalyst precursor was diluted and mixed with KBr such that the catalyst concentration was 0.5 to 1% by mass, and the resulting mixture was molded into pellets and measured in the same manner. For the thus obtained infrared absorption spectrum, the absorbance and the wavenumber were plotted on the ordinate and the abscissa, respectively, and horizontal baseline correction was performed such that the absorbance at 1,200 cm⁻¹ was 0. A Keggin structure is known to be observed with four characteristic peaks that are attributed to the vibrations of P—O, M-O, M-O-M between octahedrons, and M-O-M within octahedrons (M=Mo, V, or the like), and these peaks are observed at about 1,065 cm⁻¹, 965 cm⁻¹, 870 cm⁻¹, and 790 cm⁻¹. When all of these peaks were observed in the obtained infrared absorption spectrum, it was judged that a Keggin structure was present.

### (Analysis of Raw Material Gas and Product)

A raw material gas and a product were analyzed by gas chromatography (apparatus: GC-2014 manufactured by Shimadzu Corporation, column: DB-FFAP manufactured by J&W Scientific, Inc., 30 m×0.32 mm, membrane thickness: 1.0 μm). The yield of generated methacrylic acid was calculated by the following equation:

Yield (%) of methacrylic acid=(N2/N1)×100

Here, N1 represents the number of moles of supplied methacrolein, and N2 represents the number of moles of generated methacrylic acid.

### [Catalyst Precursor]

### Example 1-1

To 400 parts of 25° C. pure water used as a solvent, 100 parts of molybdenum trioxide, 7.5 parts of ammonium metavanadate, 11.4 parts of a 85%-by-mass aqueous phosphoric acid solution, and 7.0 parts of copper (II) nitrate trihydrate were added. The resulting slurry was heated to 95° C. with stirring, and then stirred for 3 hours with the liquid temperature being maintained at 95° C. Subsequently, while stirring the slurry with the liquid temperature being maintained at 95° C., the slurry was mixed with a solution obtained by dissolving 15.7 parts of cesium bicarbonate in 20 parts of pure water, and the resultant was stirred for 15 minutes to obtain a liquid A1.

Then, while maintaining the temperature of the liquid A1 at 95° C. and stirring the liquid A1 using a rotary blade stirrer at 110 rpm, a solution obtained by dissolving 15.0 parts of ammonium carbonate in 20 parts of pure water was added thereto. This addition required 105.7 minutes. The temperature and the v/M value of the liquid A1 in this process are shown in Table 1. After the completion of the addition, the liquid A1 was further stirred for 15 minutes with the liquid temperature being maintained at 95° C. to obtain a liquid A2. The pH, the solid concentration, and R of the thus obtained liquid A2 are shown in Table 1.

The thus obtained liquid A2 was spray-dried at a dryer inlet temperature of 300° C. to obtain a catalyst precursor. This catalyst precursor had an elemental composition of Mo₁₂P_{1.7}V_{1.1}Cu_{0.5}Cs_{1.4}(NH₄)_{5.7}, excluding oxygen. Further, this catalyst precursor contained a Keggin-type heteropolyacid. The pore volume, the median diameter, and the bulk density of the catalyst precursor are shown in Table 1.

Thereafter, 100 parts of the thus obtained catalyst precursor was mixed with 5 parts of hydroxypropylcellulose, 4 parts of pure water, and 16 parts of ethanol, and the resultant was kneaded to a clay state to obtain a mixture. Using an extrusion molding machine, this mixture was extruded at an extrusion pressure of 10 MPa and molded into a cylindrical shape of 5.5 mm in diameter and 5 mm in height, and then dried in an air stream at 25° C. for 12 hours, whereby a molded article was obtained.

The thus obtained molded article was calcined in an air atmosphere at 380° C. for 2 hours to obtain a catalyst.

The thus obtained catalyst was packed into a reaction tube to form a catalyst layer, and an oxidation reaction of methacrolein was performed under the following conditions. The results thereof are shown in Table 1.
Raw material gas composition: 5% by volume of methacrolein, 10% by volume of oxygen, 30% by volume of water vapor, and 55% by volume of nitrogen
Reaction temperature: 285° C.
Contact time between raw material gas and catalyst: 3.6 seconds

### Example 1-2

A liquid A1 was obtained in the same manner as in Example 1-1.

Subsequently, while maintaining the temperature of the liquid A1 at 95° C. and stirring the liquid A1 using a rotary blade stirrer at 110 rpm, a solution obtained by dissolving 12.3 parts of ammonium carbonate in 20 parts of pure water was added thereto. This addition required 86.6 minutes. The temperature and the v/M value of the liquid A1 in this process are shown in Table 1. After the completion of the addition, the liquid A1 was further stirred for 15 minutes with the liquid temperature being maintained at 95° C. to obtain a liquid A2. The pH, the solid concentration, and R of the thus obtained liquid A2 are shown in Table 1.

The thus obtained liquid A2 was dried in the same manner as in Example 1-1 to obtain a catalyst precursor. This catalyst precursor had an elemental composition of Mo₁₂P_{1.7}V_{1.1}Cu_{0.5}Cs_{1.4}(NH₄)_{4.9}, excluding oxygen. Further, this catalyst precursor contained a Keggin-type heteropolyacid. The pore volume, the median diameter, and the bulk density of the catalyst precursor are shown in Table 1.

The thus obtained catalyst precursor was molded and calcined in the same manner as in Example 1-1.

The thus obtained catalyst was packed into a reaction tube to form a catalyst layer, and an oxidation reaction of methacrolein was performed in the same manner as in Example 1-1. The results thereof are shown in Table 1.

### Example 1-3

A liquid A2 was obtained in the same manner as in Example 1-1. The thus obtained liquid A2 was dried in the same manner as in Example 1-1 and then sieved using a sieve having a mesh size of 15 μm, and the material fell under the sieve was obtained as a catalyst precursor. This catalyst precursor had an elemental composition of Mo₁₂P_{1.7}V_{1.1}Cu_{0.5}Cs_{1.4}(NH₄)_{5.7}, excluding oxygen. Further, this catalyst precursor contained a Keggin-type heteropolyacid. The pore volume, the median diameter, and the bulk density of the catalyst precursor are shown in Table 1.

The thus obtained catalyst precursor was molded and calcined in the same manner as in Example 1-1.

The thus obtained catalyst was packed into a reaction tube to form a catalyst layer, and an oxidation reaction of methacrolein was performed in the same manner as in Example 1-1. The results thereof are shown in Table 1.

### Example 1-4

A liquid A1 was obtained in the same manner as in Example 1-1, except that 13.5 parts of cesium carbonate was used in place of 15.7 parts of cesium bicarbonate.

Subsequently, using the liquid A1, a liquid A2 was obtained in the same manner as in Example 1-1. The pH, the solid concentration, and R of the thus obtained liquid A2 are shown in Table 1.

The thus obtained liquid A2 was spray-dried at a dryer inlet temperature of 250° C. to obtain a catalyst precursor. This catalyst precursor had an elemental composition of Mo₁₂P_{1.7}V_{1.1}Cu_{0.5}Cs_{1.4}(NH₄)_{5.7}, excluding oxygen. Further, this catalyst precursor contained a Keggin-type heteropolyacid. The pore volume, the median diameter, and the bulk density of the catalyst precursor are shown in Table 1.

The thus obtained catalyst precursor was molded and calcined in the same manner as in Example 1-1.

The thus obtained catalyst was packed into a reaction tube to form a catalyst layer, and an oxidation reaction of methacrolein was performed in the same manner as in Example 1-1. The results thereof are shown in Table 1.

### Example 1-5

A liquid A1 was obtained in the same manner as in Example 1-1.

Subsequently, a liquid A2 was obtained in the same manner as in Example 1-1, except that the addition of the solution obtained by dissolving 15.0 parts of ammonium carbonate in 20 parts of pure water required 21.2 minutes. The v/M value in this process, as well as the pH, the solid concentration, and R of the thus obtained liquid A2 are shown in Table 1.

The thus obtained liquid A2 was dried in the same manner as in Example 1-1 to obtain a catalyst precursor. This catalyst precursor had an elemental composition of Mo₁₂P_{1.7}V_{1.1}Cu_{0.5}Cs_{1.4}(NH₄)_{1.9}, excluding oxygen. Further, this catalyst precursor contained a Keggin-type heteropolyacid. The pore volume, the median diameter, and the bulk density of the catalyst precursor are shown in Table 1.

The thus obtained catalyst precursor was molded and calcined in the same manner as in Example 1-1.

The thus obtained catalyst was packed into a reaction tube to form a catalyst layer, and an oxidation reaction of methacrolein was performed in the same manner as in Example 1-1. The results thereof are shown in Table 1.

### Comparative Example 1-1

To 400 parts of 25° C. pure water used as a solvent, 100 parts of molybdenum trioxide, 7.5 parts of ammonium metavanadate, and 7.0 parts of copper (II) nitrate trihydrate were added. The resulting slurry was heated to 95° C. with stirring, and then stirred for 3 hours with the liquid temperature being maintained at 95° C. Subsequently, the slurry was cooled to a liquid temperature of 50° C. and, while stirring the slurry with the liquid temperature being maintained at 50° C., the slurry was mixed with a solution obtained by dissolving 15.7 parts of cesium bicarbonate in 20 parts of pure water, after which the resultant was stirred for 15 minutes to obtain a liquid A1′.

Then, while maintaining the temperature of the liquid A1′ at 50° C. and stirring the liquid A1′ using a rotary blade stirrer at 110 rpm, a solution obtained by dissolving 15.8 parts of ammonium nitrate in 20 parts of pure water was added thereto. This addition required 6.8 minutes. The temperature and the v/M value of the liquid A1′ in this process are shown in Table 1. After the completion of the addition, the liquid A1′ was further stirred for 15 minutes with the liquid temperature being maintained at 95° C. The resulting slurry was mixed with 11.4 parts of a 85%-by-mass aqueous phosphoric acid solution, and the resultant was stirred for 15 minutes to obtain a liquid A2′. The pH, the solid concentration, and R of the thus obtained liquid A2′ are shown in Table 1.

The thus obtained liquid A2′ was dried in the same manner as in Example 1-1 to obtain a catalyst precursor. This catalyst precursor had an elemental composition of Mo₁₂P_{1.7}V_{1.1}Cu_{0.5}Cs_{1.4}(NH₄)_{4.5}, excluding oxygen. Further, this catalyst precursor contained a Keggin-type heteropolyacid. The pore volume, the median diameter, and the bulk density of the catalyst precursor are shown in Table 1.

The thus obtained catalyst precursor was molded and calcined in the same manner as in Example 1-1.

The thus obtained catalyst was packed into a reaction tube to form a catalyst layer, and an oxidation reaction of methacrolein was performed in the same manner as in Example 1-1. The results thereof are shown in Table 1.

### Comparative Example 1-2

A liquid A1′ was obtained in the same manner as in Comparative Example 1-1, except that 200 parts of 25° C. pure water was used as a solvent.

Subsequently, using the liquid A1′, a liquid A2′ was obtained in the same manner as in Comparative Example 1-1. The pH, the solid concentration, and R of the thus obtained liquid A2′ are shown in Table 1.

The thus obtained liquid A2′ was dried in the same manner as in Example 1-1 to obtain a catalyst precursor. This catalyst precursor had an elemental composition of Mo₁₂P_{1.7}V_{1.1}Cu_{0.5}Cs_{1.4}(NH₄)_{4.5}, excluding oxygen. Further, this catalyst precursor contained a Keggin-type heteropolyacid. The pore volume, the median diameter, and the bulk density of the catalyst precursor are shown in Table 1.

The thus obtained catalyst precursor was molded and calcined in the same manner as in Example 1-1.

The thus obtained catalyst was packed into a reaction tube to form a catalyst layer, and an oxidation reaction of methacrolein was performed in the same manner as in Example 1-1. The results thereof are shown in Table 1.

### Comparative Example 1-3

A liquid A1 was obtained in the same manner as in Example 1-1.

Subsequently, a liquid A2′ was obtained in the same manner as in Example 1-1, except that the addition of the solution obtained by dissolving 15.0 parts of ammonium carbonate in 20 parts of pure water required 184.0 minutes. The v/M value in this process, as well as the pH, the solid concentration, and R of the thus obtained liquid A2′ are shown in Table 1.

The thus obtained liquid A2′ was dried in the same manner as in Example 1-1 to obtain a catalyst precursor. This catalyst precursor had an elemental composition of Mo₁₂P_{1.7}V_{1.1}Cu_{0.5}Cs_{1.4}(NH₄)_{2.0}, excluding oxygen. Further, this catalyst precursor contained a Keggin-type heteropolyacid. The pore volume, the median diameter, and the bulk density of the catalyst precursor are shown in Table 1.

The thus obtained catalyst precursor was molded and calcined in the same manner as in Example 1-1.

The thus obtained catalyst was packed into a reaction tube to form a catalyst layer, and an oxidation reaction of methacrolein was performed in the same manner as in Example 1-1. The results thereof are shown in Table 1.

TABLE 1

| | Conditions | | | | | | | | |
|---|---|---|---|---|---|---|---|---|---|
| | for adding | | Physical properties of | | | | | | |
| | compound B | | liquid A2 or liquid A2′ | | | | | | |
| | Temperature | | | Solid | | Physical properties of | | | Metha- |
| | of liquid | | | concen- | | catalyst precursor | | | crylic |
| | A1 or | | | tration | R | Pore | Median | Bulk | acid |
| | liquid | v/M | | (% by | (% by | volume | diameter | density | yield |
| | A1′(° C.) | (h⁻¹) | pH | mass) | mass) | (mL/g) | (μm) | (kg/L) | (%) |
| Example | 95 | 0.22 | 2.52 | 26.5 | 6.7 | 0.07 | 20.3 | 1.36 | 68.0 |
| 1-1 | | | | | | | | | |
| Example | 95 | 0.22 | 2.48 | 23.5 | 12.3 | 0.07 | 13.7 | 1.25 | 66.4 |
| 1-2 | | | | | | | | | |
| Example | 95 | 0.22 | 2.50 | 26.5 | 6.7 | 0.08 | 12.3 | 1.21 | 66.3 |
| 1-3 | | | | | | | | | |
| Example | 95 | 0.22 | 2.59 | 23.0 | 11.7 | 0.07 | 29.4 | 1.40 | 66.0 |
| 1-4 | | | | | | | | | |
| Example | 95 | 1.05 | 2.31 | 17.0 | 20.9 | 0.12 | 17.8 | 1.37 | 64.0 |
| 1-5 | | | | | | | | | |
| Compar- | 50 | 2.50 | 2.73 | 21.9 | 4.3 | 0.37 | 25.8 | 0.75 | 48.2 |
| ative | | | | | | | | | |
| Example | | | | | | | | | |
| 1-1 | | | | | | | | | |
| Compar- | 50 | 2.50 | 2.34 | 31.9 | 2.4 | 0.28 | 26.0 | 0.81 | 50.6 |
| ative | | | | | | | | | |
| Example | | | | | | | | | |
| 1-2 | | | | | | | | | |
| Compar- | 50 | 0.08 | 2.45 | 20.0 | 8.5 | 0.16 | 12.4 | 0.99 | 22.0 |
| ative | | | | | | | | | |
| Example | | | | | | | | | |
| 1-3 | | | | | | | | | |

As shown in Table 1, in Examples 1-1 to 1-5 where catalyst precursors having a pore volume in a prescribed range were used, a higher methacrylic acid yield was obtained as compared to Comparative Examples.

It is noted here that a methacrylic acid ester can be obtained by esterifying the methacrylic acid obtained in each of Examples 1-1 to 1-5.

### [Molded Catalyst Product]

### Example 2-1

To 400 parts of 25° C. pure water used as a solvent, 100 parts of molybdenum trioxide, 7.5 parts of ammonium metavanadate, 11.4 parts of an 85%-by-mass aqueous phosphoric acid solution, and 7.0 parts of copper (II) nitrate trihydrate were added. The resulting slurry was heated to 95° C. with stirring, and then stirred for 3 hours with the liquid temperature being maintained at 95° C. Subsequently, while stirring the slurry with the liquid temperature being maintained at 95° C., the slurry was mixed with a solution obtained by dissolving 15.7 parts of cesium bicarbonate in 20 parts of pure water, and the resultant was stirred for 15 minutes to obtain a liquid A1.

Then, while maintaining the temperature of the liquid A1 at 95° C. and stirring the liquid A1 using a rotary blade stirrer at 110 rpm, a solution obtained by dissolving 15.0 parts of ammonium carbonate in 20 parts of pure water was added thereto. This addition required 105.7 minutes. The temperature and the v/M value of the liquid A1 in this process are shown in Table 2. After the completion of the addition, the liquid A1 was further stirred for 15 minutes with the liquid temperature being maintained at 95° C. to obtain a liquid A2. The pH, the solid concentration, and R of the thus obtained liquid A2 are shown in Table 2.

The thus obtained liquid A2 was spray-dried at a dryer inlet temperature of 300° C. to obtain dry particles.

Subsequently, 100 parts of the thus obtained dry particles was mixed with 5 parts of hydroxypropylcellulose, 4 parts of pure water, and 16 parts of ethanol, and the resultant was kneaded to a clay state to obtain a mixture. Using an extrusion molding machine, this mixture was extruded at an extrusion pressure of 10 MPa and molded into a cylindrical shape of 5.5 mm in diameter and 5 mm in height. This molded product was dried in an air stream at 25° C. for 12 hours, and then calcined in an air atmosphere at 380° C. for 2 hours, whereby a molded catalyst product was obtained. This molded catalyst product had an elemental composition of Mo₁₂P_{1.7}V_{1.1}Cu_{0.5}Cs_{1.4} excluding oxygen, and an ammonium radical molar ratio of 1 or lower. Further, the molded catalyst product had two peaks in a pore diameter range of 0.05 to 10 μm in its pore distribution curve. The specific surface area, the pore volume, and the ratio IB/IA in the pore distribution curve of the molded catalyst product are shown in Table 2.

Thereafter, the thus obtained molded catalyst product was packed into a reaction tube to form a molded catalyst product layer, and an oxidation reaction of methacrolein was performed under the following conditions. The results thereof are shown in Table 2.

Raw material gas composition: 5% by volume of methacrolein, 10% by volume of oxygen, 30% by volume of water vapor, and 55% by volume of nitrogen
Reaction temperature: 285° C.
Contact time between raw material gas and molded catalyst product: 3.6 seconds

### Example 2-2

A liquid A1 was obtained in the same manner as in Example 2-1.

Subsequently, while maintaining the temperature of the liquid A1 at 95° C. and stirring the liquid A1 using a rotary blade stirrer at 110 rpm, a solution obtained by dissolving 12.3 parts of ammonium carbonate in 20 parts of pure water was added thereto. This addition required 86.6 minutes. The temperature and the v/M value of the liquid A1 in this process are shown in Table 2. After the completion of the addition, the liquid A1 was further stirred for 15 minutes with the liquid temperature being maintained at 95° C. to obtain a liquid A2. The pH, the solid concentration, and R of the thus obtained liquid A2 are shown in Table 2.

The thus obtained liquid A2 was spray-dried at the same dryer inlet temperature as in Example 2-1 to obtain dry particles.

Subsequently, the thus obtained dry particles were molded, dried, and calcined in the same manner as in Example 2-1 to obtain a molded catalyst product. This molded catalyst product had an elemental composition of Mo₁₂P_{1.7}V_{1.1}Cu_{0.5}Cs_{1.4} excluding oxygen, and an ammonium radical molar ratio of 1 or lower. Further, the molded catalyst product had two peaks in a pore diameter range of 0.05 to 10 μm in its pore distribution curve. The specific surface area, the pore volume, and the ratio IB/IA in the pore distribution curve of the molded catalyst product are shown in Table 2.

Thereafter, the thus obtained molded catalyst product was packed into a reaction tube to form a molded catalyst product layer, and an oxidation reaction of methacrolein was performed in the same manner as in Example 2-1. The results thereof are shown in Table 2.

### Example 2-3

A liquid A2 was obtained in the same manner as in Example 2-1.

The thus obtained liquid A2 was spray-dried at the same dryer inlet temperature as in Example 2-1 and then sieved using a sieve having a mesh size of 15 μm, and the material fell under the sieve was obtained as dry particles.

Subsequently, the thus obtained dry particles were molded, dried, and calcined in the same manner as in Example 2-1 to obtain a molded catalyst product. This molded catalyst product had an elemental composition of Mo₁₂P_{1.7}V_{1.1}Cu_{0.5}Cs_{1.4} excluding oxygen, and an ammonium radical molar ratio of 1 or lower. Further, the molded catalyst product had two peaks in a pore diameter range of 0.05 to 10 μm in its pore distribution curve. The specific surface area, the pore volume, and the ratio IB/IA in the pore distribution curve of the molded catalyst product are shown in Table 2.

Thereafter, the thus obtained molded catalyst product was packed into a reaction tube to form a molded catalyst product layer, and an oxidation reaction of methacrolein was performed in the same manner as in Example 2-1. The results thereof are shown in Table 2.

### Example 2-4

A liquid A1 was obtained in the same manner as in Example 2-1, except that 13.5 parts of cesium carbonate was used in place of 15.7 parts of cesium bicarbonate.

Subsequently, using the liquid A1, a liquid A2 was obtained in the same manner as in Example 2-1. The pH, the solid concentration, and R of the thus obtained liquid A2 are shown in Table 2.

The thus obtained liquid A2 was spray-dried at a dryer inlet temperature of 250° C. to obtain dry particles.

Subsequently, the thus obtained dry particles were molded, dried, and calcined in the same manner as in Example 2-1 to obtain a molded catalyst product. This molded catalyst product had an elemental composition of Mo₁₂P_{1.7}V_{1.1}Cu_{0.5}Cs_{1.4} excluding oxygen, and an ammonium radical molar ratio of 1 or lower. Further, the molded catalyst product had two peaks in a pore diameter range of 0.05 to 10 μm in its pore distribution curve. The specific surface area, the pore volume, and the ratio IB/IA in the pore distribution curve of the molded catalyst product are shown in Table 2.

Thereafter, the thus obtained molded catalyst product was packed into a reaction tube to form a molded catalyst product layer, and an oxidation reaction of methacrolein was performed in the same manner as in Example 2-1. The results thereof are shown in Table 2.

### Example 2-5

A liquid A1 was obtained in the same manner as in Example 2-1.

Subsequently, a liquid A2 was obtained in the same manner as in Example 2-1, except that the addition of the solution obtained by dissolving 15.0 parts of ammonium carbonate in 20 parts of pure water required 21.2 minutes. The v/M value in this process, as well as the pH, the solid concentration, and R of the thus obtained liquid A2 are shown in Table 2.

The thus obtained liquid A2 was dried in the same manner as in Example 2-1 to obtain dry particles.

Subsequently, the thus obtained dry particles were molded, dried, and calcined in the same manner as in Example 2-1 to obtain a molded catalyst product. This molded catalyst product had an elemental composition of Mo₁₂P_{1.7}V_{1.1}Cu_{0.5}Cs_{1.4} excluding oxygen, and an ammonium radical molar ratio of 1 or lower. Further, the molded catalyst product had two peaks in a pore diameter range of 0.05 to 10 μm in its pore distribution curve. The specific surface area, the pore volume, and the ratio IB/IA in the pore distribution curve of the molded catalyst product are shown in Table 2.

Thereafter, the thus obtained molded catalyst product was packed into a reaction tube to form a molded catalyst product layer, and an oxidation reaction of methacrolein was performed in the same manner as in Example 2-1. The results thereof are shown in Table 2.

### Comparative Example 2-1

To 400 parts of 25° C. pure water used as a solvent, 100 parts of molybdenum trioxide, 7.5 parts of ammonium metavanadate, and 7.0 parts of copper (II) nitrate trihydrate were added. The resulting slurry was heated to 95° C. with stirring, and then stirred for 3 hours with the liquid temperature being maintained at 95° C. Subsequently, the slurry was cooled to a liquid temperature of 50° C. and, while stirring the slurry with the liquid temperature being maintained at 50° C., the slurry was mixed with a solution obtained by dissolving 15.7 parts of cesium bicarbonate in 20 parts of pure water, after which the resultant was stirred for 15 minutes to obtain a liquid A1′.

Then, while maintaining the temperature of the liquid A1′ at 50° C. and stirring the liquid A1′ using a rotary blade stirrer at 110 rpm, a solution obtained by dissolving 15.8 parts of ammonium nitrate in 20 parts of pure water was added thereto. This addition required 6.8 minutes. The temperature and the v/M value of the liquid A1′ in this process are shown in Table 2. After the completion of the addition, the liquid A1′ was further stirred for 15 minutes with the liquid temperature being maintained at 95° C. The resulting slurry was mixed with 11.4 parts of an 85%-by-mass aqueous phosphoric acid solution, and the resultant was stirred for 15 minutes to obtain a liquid A2′. The pH, the solid concentration, and R of the thus obtained liquid A2′ are shown in Table 2.

The thus obtained liquid A2′ was spray-dried at the same dryer inlet temperature as in Example 2-1 to obtain dry particles.

Subsequently, the thus obtained dry particles were molded, dried, and calcined in the same manner as in Example 2-1 to obtain a molded catalyst product. This molded catalyst product had an elemental composition of Mo₁₂P_{1.7}V_{1.1}Cu_{0.5}Cs_{1.4} excluding oxygen, and an ammonium radical molar ratio of 1 or lower. Further, the molded catalyst product had two peaks in a pore diameter range of 0.05 to 10 μm in its pore distribution curve. The specific surface area, the pore volume, and the ratio IB/IA in the pore distribution curve of the molded catalyst product are shown in Table 2.

Thereafter, the thus obtained molded catalyst product was packed into a reaction tube to form a molded catalyst product layer, and an oxidation reaction of methacrolein was performed in the same manner as in Example 2-1. The results thereof are shown in Table 2.

### Comparative Example 2-2

A liquid A1′ was obtained in the same manner as in Comparative Example 2-1, except that 200 parts of 25° C. pure water was used as a solvent.

Subsequently, using the liquid A1′, a liquid A2′ was obtained in the same manner as in Comparative Example 2-1. The pH, the solid concentration, and R of the thus obtained liquid A2′ are shown in Table 2.

The thus obtained liquid A2′ was spray-dried at the same dryer inlet temperature as in Example 2-1 to obtain dry particles.

Subsequently, the thus obtained dry particles were molded, dried, and calcined in the same manner as in Example 2-1 to obtain a molded catalyst product. This molded catalyst product had an elemental composition of Mo₁₂P_{1.7}V_{1.1}Cu_{0.5}Cs_{1.4} excluding oxygen, and an ammonium radical molar ratio of 1 or lower. Further, the molded catalyst product had two peaks in a pore diameter range of 0.05 to 10 μm in its pore distribution curve. The specific surface area, the pore volume, and the ratio IB/IA in the pore distribution curve of the molded catalyst product are shown in Table 2.

Thereafter, the thus obtained molded catalyst product was packed into a reaction tube to form a molded catalyst product layer, and an oxidation reaction of methacrolein was performed in the same manner as in Example 2-1. The results thereof are shown in Table 2.

### Comparative Example 2-3

A liquid A1 was obtained in the same manner as in Example 2-1.

Subsequently, while maintaining the temperature of the liquid A1 at 95° C. and stirring the liquid A1 using a rotary blade stirrer at 110 rpm, a solution obtained by dissolving 15.0 parts of ammonium carbonate in 20 parts of pure water was added thereto. This addition required 105.7 minutes. The temperature and the v/M value of the liquid A1 in this process are shown in Table 2. After the completion of the addition, the liquid A1 was further stirred for 15 minutes with the liquid temperature being maintained at 95° C. and then heated to 100° C. Thereafter, the liquid A1 was stirred for 40 minutes with the liquid temperature being maintained at 100° C., whereby a liquid A2 was obtained. The pH, the solid concentration, and R of the thus obtained liquid A2 are shown in Table 2.

The thus obtained liquid A2 was dried at 140° C. using a drum dryer to obtain dry particles.

Subsequently, the thus obtained dry particles were molded, dried, and calcined in the same manner as in Example 2-1 to obtain a molded catalyst product. This molded catalyst product had an elemental composition of Mo₁₂P_{1.7}V_{1.1}Cu_{0.5}Cs_{1.4} excluding oxygen, and an ammonium radical molar ratio of 1 or lower. Further, the molded catalyst product had two peaks in a pore diameter range of 0.05 to 10 μm in its pore distribution curve. The specific surface area, the pore volume, and the ratio IB/IA in the pore distribution curve of the molded catalyst product are shown in Table 2.

Thereafter, the thus obtained molded catalyst product was packed into a reaction tube to form a molded catalyst product layer, and an oxidation reaction of methacrolein was performed in the same manner as in Example 2-1. The results thereof are shown in Table 2.

### Comparative Example 2-4

A liquid A1 was obtained in the same manner as in Example 2-1, except that the solution obtained by dissolving 15.7 parts of cesium bicarbonate in 20 parts of pure water was mixed while stirring the slurry with the liquid temperature being maintained at 50° C.

Subsequently, a liquid A2′ was obtained in the same manner as in Example 2-1, except that the addition of the solution obtained by dissolving 15.0 parts of ammonium carbonate in 20 parts of pure water required 184.0 minutes. The v/M value in this process, as well as the pH, the solid concentration, and R of the thus obtained liquid A2′ are shown in Table 2.

The thus obtained liquid A2′ was dried in the same manner as in Example 2-1 to obtain dry particles.

Subsequently, the thus obtained dry particles were molded, dried, and calcined in the same manner as in Example 2-1 to obtain a molded catalyst product. This molded catalyst product had an elemental composition of Mo₁₂P_{1.7}V_{1.1}Cu_{0.5}Cs_{1.4} excluding oxygen, and an ammonium radical molar ratio of 1 or lower. Further, the molded catalyst product had two peaks in a pore diameter range of 0.05 to 10 μm in its pore distribution curve. The specific surface area, the pore volume, and the ratio IB/IA in the pore distribution curve of the molded catalyst product are shown in Table 2.

Thereafter, the thus obtained molded catalyst product was packed into a reaction tube to form a molded catalyst product layer, and an oxidation reaction of methacrolein was performed in the same manner as in Example 2-1. The results thereof are shown in Table 2.

TABLE 2

| | Conditions | | | | | | | | |
|---|---|---|---|---|---|---|---|---|---|
| | for adding | | Physical properties of | | | | | | |
| | compound B | | liquid A2 or liquid A2′ | | | Physical properties of | | | |
| | Temperature | | | Solid | | molded catalyst product | | | Metha- |
| | of liquid | | | concen- | | | | Specific | crylic |
| | A1 or | | | tration | R | Pore | | surface | acid |
| | liquid | v/M | | (% by | (% by | volume | | area | yield |
| | A1′(° C.) | (h⁻¹) | pH | mass) | mass) | (mL/g) | IB/IA | (m²/g) | (%) |
| Example | 95 | 0.22 | 2.52 | 26.5 | 6.7 | 0.25 | 0.397 | 3 | 68.0 |
| 2-1 | | | | | | | | | |
| Example | 95 | 0.22 | 2.48 | 23.5 | 12.3 | 0.20 | 0.227 | 3 | 66.4 |
| 2-2 | | | | | | | | | |
| Example | 95 | 0.22 | 2.50 | 26.5 | 6.7 | 0.24 | 0.218 | 3 | 66.3 |
| 2-3 | | | | | | | | | |
| Example | 95 | 0.22 | 2.59 | 23.0 | 11.7 | 0.21 | 0.398 | 3 | 66.0 |
| 2-4 | | | | | | | | | |
| Example | 95 | 1.09 | 2.31 | 17.0 | 20.9 | 0.20 | 0.310 | 5 | 64.0 |
| 2-5 | | | | | | | | | |
| Compar- | 50 | 2.50 | 2.73 | 21.9 | 4.3 | 0.61 | 0.056 | 11 | 48.2 |
| ative | | | | | | | | | |
| Example | | | | | | | | | |
| 2-1 | | | | | | | | | |
| Compar- | 50 | 2.50 | 2.34 | 31.9 | 2.4 | 0.54 | 0.154 | 8 | 50.6 |
| ative | | | | | | | | | |
| Example | | | | | | | | | |
| 2-2 | | | | | | | | | |
| Compar- | 95 | 0.22 | 2.61 | 36.5 | 6.7 | 0.22 | 0.426 | 6 | 57.9 |
| ative | | | | | | | | | |
| Example | | | | | | | | | |
| 2-3 | | | | | | | | | |
| Compar- | 50 | 0.08 | 2.45 | 20.0 | 8.5 | 0.40 | 0.850 | 2 | 22.0 |
| ative | | | | | | | | | |
| Example | | | | | | | | | |
| 2-4 | | | | | | | | | |

As shown in Table 2, in Examples 2-1 to 2-5 where the pore volume and the ratio IB/IA in the pore distribution curve of the respective molded catalyst products were in prescribed ranges, a higher methacrylic acid yield was obtained as compared to Comparative Examples.

It is noted here that a methacrylic acid ester can be obtained by esterifying the methacrylic acid obtained in each of Examples 2-1 to 2-5.

### INDUSTRIAL APPLICABILITY

According to the present invention, a catalyst precursor from which a catalyst capable of producing an α,β-unsaturated carboxylic acid with a high yield can be produced, and a molded catalyst product can be provided.

What is claimed is:

1. A catalyst precursor, comprising a Keggin-type heteropolyacid,
wherein the catalyst precursor has a pore volume of 0.005 to 0.15 mL/g.

2. The catalyst precursor according to claim 1, having a median diameter of 1 to 50 μm.

3. The catalyst precursor according to claim 2, wherein the median diameter is 5 to 40 μm.

4. The catalyst precursor according to claim 1, wherein the pore volume is 0.01 to 0.10 mL/g.

5. The catalyst precursor according to claim 1, having a bulk density of 1.15 to 1.6 kg/L.

6. The catalyst precursor according to claim 1, having a composition represented by the following Formula (I):
PₐMO_{b}V_{c}Cu_{d}AₑE_{f}G_{g}(NH₄)ₕOᵢ (I)
wherein, P, Mo, V, Cu, NH₄, and O represent phosphorus, molybdenum, vanadium, copper, ammonium radical, and oxygen, respectively; A represents at least one element selected from the group consisting of antimony, bismuth, arsenic, germanium, tellurium, selenium, silicon, and tungsten; E represents at least one element selected from the group consisting of iron, zinc, chromium, tantalum, cobalt, nickel, manganese, titanium, niobium, and cerium; G represents at least one element selected from the group consisting of lithium, sodium, potassium, rubidium, and cesium; and a to i each represent a molar ratio of the respective component, satisfying b=12, a=0.5 to 3, c=0.01 to 3, d=0.01 to 2, e=0 to 3, f=0 to 3, g=0.01 to 3, and h=1 to 30, with i being a molar ratio of oxygen that is required for satisfying the valences of the respective elements.

7. A molded catalyst product, comprising a catalyst component containing phosphorus, molybdenum, and vanadium,
wherein
the molded catalyst product has a pore volume of 0.01 to 0.40 mL/g, and
in a pore distribution curve of the molded catalyst product, when a height of a highest peak (peak A) is IA, a height of a second highest peak (peak B) is IB, and apexes of the peak A and the peak B exist in a pore diameter range of 0.05 to 10 μm, a ratio IB/IA is 0.160 to 0.420.

8. The molded catalyst product according to claim 7, wherein the ratio IB/IA is 0.200 to 0.400.

9. The molded catalyst product according to claim 7, having a specific surface area of 1 to 10 m²/g.

10. The molded catalyst product according to claim 9, wherein the specific surface area is 1.5 to 8 m²/g.

11. The molded catalyst product according to claim 7, wherein the pore volume is 0.10 to 0.35 mL/g.

12. The molded catalyst product according to claim 7, wherein the apexes of the peak A and the peak B exist in a pore diameter range of 0.08 to 8 μm.

13. The molded catalyst product according to claim 7, which is an extrusion-molded article.

14. A method of producing the catalyst precursor according to claim 1, comprising:
obtaining a solution or slurry (liquid A1) containing phosphorus, molybdenum, and vanadium;
adding an ammonium radical-containing raw material compound (compound B) to the liquid A1 to obtain a slurry having a pH of 3 or lower (liquid A2); and
(drying the liquid A2 to obtain dry particles,
wherein, in the adding, the compound B is added such that the following Formula (II) is satisfied:
$$v / M = 0.1 to 1.8 \qquad ( II )$$
wherein, M represents a number of moles (mol) of molybdenum contained in the liquid A1, and v represents an ammonium radical addition rate (mol/h).

15. The method according to claim 14, further comprising extrusion-molding the dry particles to obtain the molded catalyst product.

16. The method according to claim 14, further comprising calcinating the catalyst precursor.

17. A method of producing the molded catalyst product according to claim 7, comprising:
obtaining a solution or slurry (liquid A1) that contains phosphorus, molybdenum, and vanadium;
adding an ammonium radical-containing raw material compound (compound B) to the liquid A1 to obtain a slurry having a pH of 3 or lower (liquid A2);
drying the liquid A2 to obtain dry particles; and
(extrusion-molding the dry particles to obtain the molded catalyst product,
wherein, in the adding, the compound B is added such that the following Formula (II) is satisfied:
$$v / M = 0.1 to 1.8 \qquad ( II )$$
wherein, M represents the number of moles (mol) of molybdenum contained in the liquid A1, and v represents an ammonium radical addition rate (mol/h).

18. The method of producing a molded catalyst product according to claim 15, wherein the liquid A2 has a solid concentration of 30% by mass or lower.

19. The method of producing a molded catalyst product according to claim 15, wherein, in the liquid A2, when a ratio of a total mass of dissolved molybdenum element, phosphorus element, and vanadium element with respect to a total mass of molybdenum element, phosphorus element, and vanadium element is R, R is 5 to 25% by mass.

20. The method of producing a molded catalyst product according to claim 15, wherein, in the adding, the compound B is added while stirring the liquid A1 having a temperature of 90 to 99° C. at a rotation speed of 70 to 140 rpm.

21. The method of producing a molded catalyst product according to claim 15, wherein, in the drying, the liquid A2 is spray-dried.

22. The method of producing a molded catalyst product according to claim 15, wherein the extrusion-molding comprises):
mixing the dry particles with a liquid and a binder to obtain a kneaded product; and
extrusion-molding the kneaded product using an extruder to obtain the molded catalyst product.

23. The method of producing a molded catalyst product according to claim 22, wherein, in the mixing, 15 to 60 parts by mass of the liquid and 0.05 to 15 parts by mass of the binder are mixed with respect to 100 parts by mass of the dry particles.

24. The method of producing a molded catalyst product according to claim 22, wherein, in the extrusion-molding the kneaded product, the extrusion molding is performed at an extrusion pressure of 0.1 to 30 MPa.

25. A method of producing an α,β-unsaturated carboxylic acid, comprising oxidizing an α,β-unsaturated aldehyde using a catalyst obtained by molding and/or calcinating the catalyst precursor according to claim 1.

26. A method of producing an α,β-unsaturated carboxylic acid ester, the method comprising esterifying α,β-unsaturated carboxylic acid produced by the method according to claim 25.
